(19) United States
(12) Patent Application Publication
(10) Pub. No.: US 2009/0192401 A1
(43) Pub. Date: Jul. 30, 2009

(54) METHOD AND SYSTEM FOR HEART SOUND IDENTIFICATION

(76) Inventor: Sourabh Ravindran, Dallas, TX (US)

Correspondence Address: TEXAS INSTRUMENTS INCORPORATED, P O BOX 655474, M/S 3999, DALLAS, TX, 75265, US

(21) Appl. No.: 12/044,807

(22) Filed: Mar. 7, 2008

Related U.S. Application Data
(60) Provisional application No. 61/023,581, filed on Jan. 25, 2008.

(51) Int. Cl. A61B 7/00

(52) U.S. Cl. 600/528

(57) ABSTRACT

Methods, systems, and computer readable media are provided for identification of heart sound components in an audio signal of heart sounds. Time domain kurtosis and frequency domain kurtosis are used to distinguish peaks corresponding to the primary heart sounds, S₁ and S₂, from murmur peaks. Timing based error correction may also be used to verify that appropriate peaks corresponding to the primary heart sounds are identified.

### CROSS REFERENCE TO RELATED APPLICATIONS

The present application claims priority to U.S. Provisional Patent Application No. 61/023,581, filed on Jan. 25, 2008, entitled “Robust Heart Rate Detection In The Presence of Pathological Conditions.”

### BACKGROUND OF THE INVENTION

Auscultation, the act of listening to the sounds of internal organs, is a valuable and simple diagnostic tool for detecting heart dysfunction, because of its non-invasive ability to provide useful information concerning the integrity and function of the heart valves and also on the hemodynamics of the heart. But, a disturbing percentage of medical graduates cannot properly diagnose heart conditions using a stethoscope. The art of listening to heart sounds and interpreting their meaning is difficult to master as the sounds are the result of several events of short duration that occur in a very small interval of time. The poor sensitivity of human ears in the low frequency range, the range in which the heart sounds occur, makes this task even more difficult.

Augmenting the information available to the physician with automatic auscultation (e.g., computer-aided auscultation using digital signal processing techniques to display a representation of heart sounds along with diagnostic information) may greatly improve the chances of correct diagnosis and avoid the need for costly screening tests. The aim of automatic auscultation is not necessarily to replace the human expert but to provide auxiliary information to help the human expert make an informed decision. An important part of automatic auscultation is the robust detection of heart rate and the location of primary heart sounds.

In automatic auscultation, heart sounds may be recorded using a diagnostic sound recording device such as an electronic stethoscope and displayed graphically in a phonocardiogram (PCG), in which the x-axis represents time and the y-axis represents a measure of the intensity of sound, i.e., amplitude. The audio signal resulting from a recording of heart sounds is a multi-component signal that includes primary heart sound components and abnormal components. The primary heart sound components, S₁ and S₂, are composite acoustic signals generated by valve closures (i.e., S₁ is caused by the closure of the mitral and tricuspid values and S₂ is caused by the closing of the aortic and pulmonary valves). The abnormal components may be clicks, snaps, and murmurs (i.e., noises associated with the damage of valves and improper functioning of valves), which can indicate abnormalities in heart structures. Two other components may also be present in the heart sounds, S₃ and S₄. S₃ occurs at the beginning of diastole just after S₂ and may, in some cases, be an indication of an abnormality. S₄ occurs at the beginning of systole just before S₁, and may also, in some cases, be an indication of an abnormality.

The localization of the abnormal components indicates different dysfunctional causes. For example, the diagnosis of heart valve disorders is based on the presence of different kind of murmurs in the cardiac cycle. A cardiac cycle is delimited by a single systole and a single diastole. Some of the features indicative of different types of murmurs include the location of the murmur, i.e., whether the murmur is present in systole or diastole, the intensity of murmur relative to the primary heart sound components, and the shape of the murmur. Accordingly, the major components of the cardiac cycle need to be separated to aid in diagnosis.

Segmentation of heart sounds into associated cardiac cycles and the detection of the location of S₁ and S₂ is a primary step prior to the automated analysis of heart sounds for diagnostic purposes. Thus, robust detection and segmentation of heart sounds is needed for automatic auscultation. Various approaches for heart sound segmentation have been proffered including using a reference electrocardiogram (ECG) signal or/and carotid pulse, using PCG signals only in the time and/or frequency domains, or using wavelet transform. More specifically, in one known segmentation approach, an adaptive tracking algorithm based on wavelet transform is used. This approach relies on information regarding the physical position of the recording to identify S₁. Further, this approach, although robust to high-frequency noise, may cause false detection when noises overlap in frequency.

In another known segmentation approach, the audio signal is filtered to suppress high frequency murmurs and then the peaks of the energy profile are picked to locate S₁ and S₂. This approach requires the heart rate be known and used as auxiliary input to detect the S₁ and S₂ locations. Further, filtering can be detrimental in detection of clicks and snaps that occur very close to S₁ and S₂. In addition, this approach may not perform well when there is spectral overlap between S₁ and S₂ and pathological conditions with high energy content. In yet another known segmentation approach, ECG signals are used to perform segmentation. In this approach, the Shannon energy measure is used to segment S₁ and S₂. Again, this approach may not perform well when there is overlap between the primary heart sounds and murmurs.

### SUMMARY OF THE INVENTION

Embodiments of the invention provide methods, systems, and computer readable media for heart sound identification. Embodiments provide for the location of the primary heart sounds, S₁ and S₂, in an audio signal of heart sounds in a manner that is robust in the presence of pathological heart conditions such as rumbles, murmurs, clicks, and snaps. Kurtosis in the time domain is used to distinguish an S₁ or S₂ peak from some types of murmur peaks and kurtosis in the frequency domain is used to distinguish an S₂ peak from peaks associated with a late systolic murmur. In addition, in some embodiments, timing based error correction is applied to help insure that the peaks selected for S₁ and S₂ are appropriate. Further, some embodiments include heart rate detection that is computationally inexpensive and works for a wide range of heart rates. In addition, some embodiments include diagnostic support for identifying pathological heart conditions indicated in the audio signal.

### BRIEF DESCRIPTION OF THE DRAWINGS

Particular embodiments in accordance with the invention will now be described, by way of example only, and with reference to the accompanying drawings:

FIGS. 1 and 2 show systems for identification of heart sounds in accordance with one or more embodiments of the invention;

FIGS. 3-6 show flow diagrams of methods for identification of heart sounds in accordance with one or more embodiments of the invention;

FIGS. 7-18 show example phonocardiograms in accordance with one or more embodiments of the invention; and

FIG. 19 shows an illustrative computer system in accordance with one or more embodiments.

### DETAILED DESCRIPTION OF EMBODIMENTS OF THE INVENTION

Specific embodiments of the invention will now be described in detail with reference to the accompanying figures. Like elements in the various figures are denoted by like reference numerals for consistency.

In the following detailed description of embodiments of the invention, numerous specific details are set forth in order to provide a more thorough understanding of the invention. However, it will be apparent to one of ordinary skill in the art that the invention may be practiced without these specific details. In other instances, well-known features have not been described in detail to avoid unnecessarily complicating the description.

In general, embodiments of the invention provide for robust identification of the primary heart sounds S₁ and S₂ in the presence of pathological conditions such as diastolic rumble, systolic murmurs, ejection clicks, etc. The primary heart sounds may be located even when a pathological heart condition masks one or both of the primary heart sounds and for a wide range of heart rates (e.g., 38 to 300 beats per minute (BPM)). More specifically, in one or more embodiments of the invention, in an audio signal of heart sounds, the locations of peaks corresponding to S₁ and S₂ in each cardiac cycle in the signal are identified. Further, kurtosis in the time domain is used to distinguish the S₁ peaks and the S₂ peaks from the peaks of some types of murmurs. In addition, kurtosis in the frequency domain may be used to distinguish the S₂ peaks from the peaks of a late systolic murmur and/or the presence of S₃ peaks. In some embodiments of the invention, timing based error correction is used to further ensure that peak locations selected for S₁ and S₂ are appropriate.

In some embodiments of the invention, after all of the S₁ and S₂ peaks in the audio signal are located, the heart rate may be determined based on the number of S₁ peaks located and the sampling frequency. Further, in one or more embodiments of the invention, the locations of the S₁ and S₂ peaks may be used in conjunction with information about the location of murmurs found while identifying S₁ and S₂ and information regarding the correction of S₁ and/or S₂ peaks during timing based error correction to provide additional diagnostic information for the classification of murmurs and other pathological conditions indicated by the heart sounds. An annotated graphical representation of the heart sounds (i.e., a phonocardiograph) that shows the locations of the S₁ and S₂ peaks may also be displayed. In some embodiments of the invention, the heart rate may and/or any additional diagnostic information regarding pathological conditions found in the heart sounds may also be displayed.

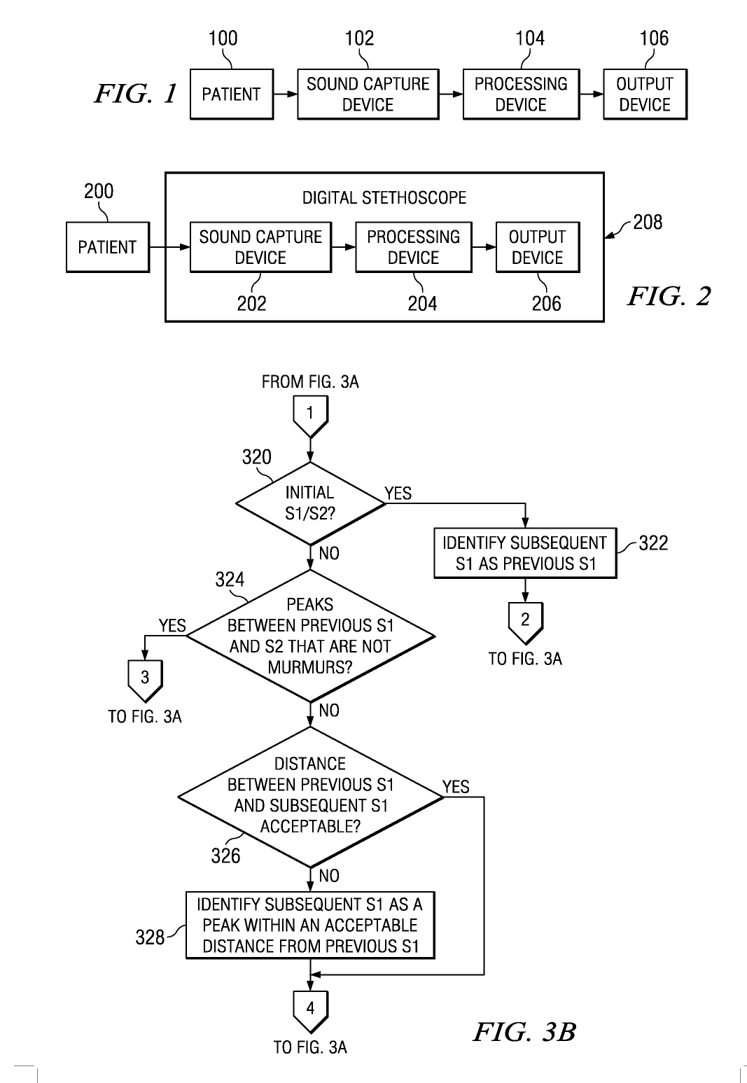

FIGS. 1 and 2 show systems for the identification of heart sounds in accordance with one or more embodiments of the invention. The system of FIG. 1 includes a sound capture device (102), a processing device (104), and an output device (106). While each of these devices is depicted and described separately, one of ordinary skill in the art will know that any two or all three of the devices may be combined in a single computing system. The sound capture device (102) is configured to capture heart sounds from a patient (100) and provide the captured heart sounds to the processing device (104) as an audio signal. More specifically, the sound capture device (102) may include functionality to convert the acoustic sound waves of the patient's (100) heart sounds to a digital audio signal. The digital audio signal may be stored by the sound capture device (102) until requested by the processing device (104) or may be provided to the processing device (104) continuously (e.g., by direct audio output) as the heart sounds are captured. In some embodiments of the invention, the sound capture device (102) may include functionality to amplify the digital audio signal and/or perform other optimizations (e.g., noise reduction) on the digital audio signal before providing the signal to the processing device (104). In one or more embodiments of the invention, the sound capture device (102) is an electronic stethoscope (i.e., stethophone).

The transmission of the digital audio signal to the processing device (104) may be wired or wireless. More specifically, the sound capture device (102) may be directly connected to the processing device (104) (e.g., using a USB port) or may be communicatively coupled to the processing device (104) by a network (not specifically shown). The network may be a wide area network (WAN) such as the Internet, a wireless network, a local area network (LAN), or a combination of networks.

The processing device (104) is a computing system (e.g., a microprocessor, a personal computer, a laptop computer, a server, a mainframe, a personal digital assistant, a television, a mobile phone, an iPod, an MP3 player, etc.) configured to receive the digital audio signal from the sound capture device (102) and to process the signal to identify the primary heart sounds, S₁ and S₂, in each cardiac cycle recorded in the signal. The processing device (104) may also be configured to determine the heart rate once the primary heart sounds are identified. Further, the processing device (104) may be configured to provide additional diagnostic information regarding pathological conditions present in the recorded heart sounds. The processing device also includes functionality to generate an annotated PCG of the digital signal and provide the PCG to the output device (106) for display. The annotations in the PCG may include locations of S₁ and S₂, the heart rate, and/or the additional diagnostic information. More specifically, the processing device includes functionality to store executable instructions implementing a method for heart sound identification as described herein and to execute those instructions.

The transmission of the PCG to the output device (106) may be wired or wireless. More specifically, the output device (106) may be directly connected to the processing device (104) (e.g., using a USB port, a controller card, control circuitry, etc.) or may be communicatively coupled to the processing device (104) by a network (not specifically shown). The network may be a wide area network (WAN) such as the Internet, a wireless network, a local area network (LAN), or a combination of networks.

The output device (106) is configured to receive the PCG from the processing device (104) and to display the PCG. The output device (106) may be any display device capable of displaying the PCG such as, for example, a computer monitor, a display screen of a handheld computing device, etc. The output device (106) may also be another computing system that includes a display device.

The system of FIG. 2 shows a digital stethoscope (208) configured to identify heart sounds in accordance with methods described herein. The digital stethoscope (208) includes a sound capture device (202), a processing device (204), and an output device (206). The sound capture device (202) is configured to capture acoustic heart sounds from a patient (200) and provide the captured heart sounds to the processing device (204) as an audio signal. The sound capture device (202) may be circuitry in a chest piece of the digital stethoscope and/or in the body of the digital stethoscope (208). More specifically, the sound capture device (202) may include functionality to convert the acoustic sound waves of the patient's (200) heart sounds to a digital audio signal that is provided to the processing device (204) continuously (e.g., by direct audio output) as the heart sounds are captured. In some embodiments of the invention, the sound capture device (202) may include functionality to amplify the digital audio signal and/or perform other optimizations (e.g., noise reduction) on the digital audio signal before providing the signal to the processing device (204).

The processing device (204) is one or more processors configured to receive the digital audio signal from the sound capture device (202). More specifically, the processing device may be a digital signal processor (DSP), a microprocessor, or a combination of a DSP and a microprocessor. The processing device (204) is further configured to process the signal to identify the primary heart sounds, S1 and S₂, in each cardiac cycle recorded in the signal. The processing device (204) may also be configured to determine the heart rate once the primary heart sounds are identified. Further, the processing device (204) may be configured to provide additional diagnostic information regarding pathological conditions present in the recorded heart sounds. The processing device also includes functionality to generate an annotated PCG of the digital signal and provide the PCG to the output device (206) for display. The annotations in the PCG may include locations of S₁ and S₂, the heart rate, and/or the additional diagnostic information. More specifically, the processing device (204) includes functionality to store executable instructions implementing a method for heart sound identification as described herein and to execute those instructions.

The output device (206) is a display screen included in the body of the digital stethoscope (208) and operatively connected to the processing device (204) by control circuitry. Further, the output device (206) is configured to receive the PCG from the processing device (204) and to display the PCG.

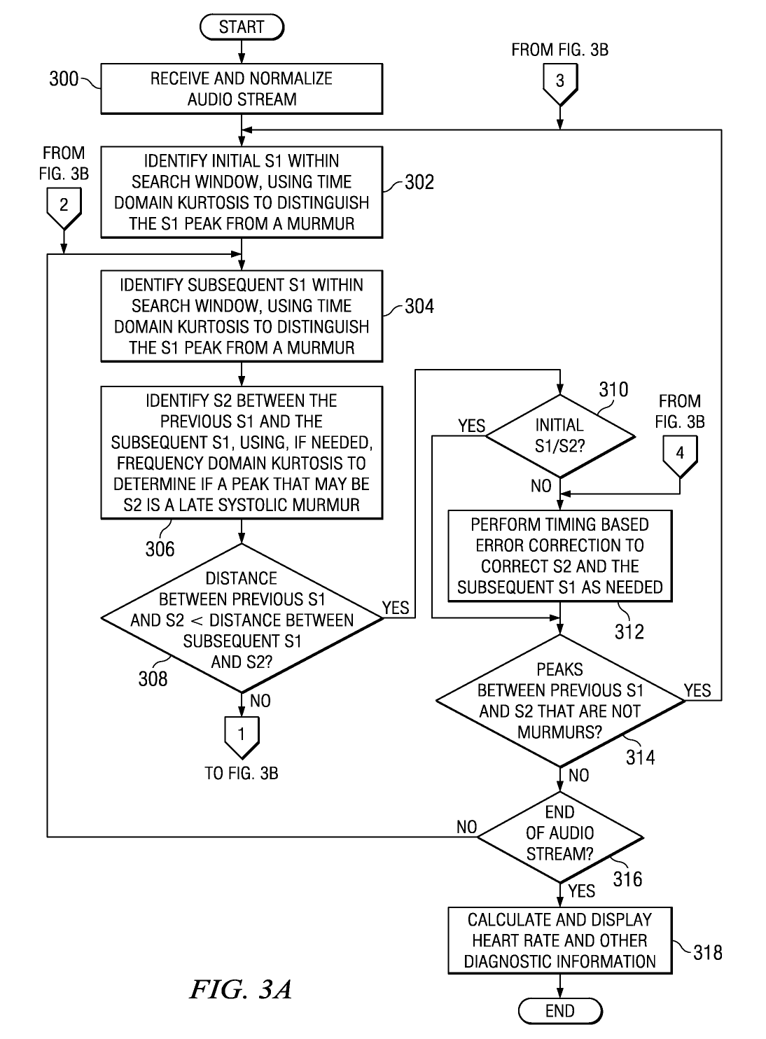

FIGS. 3-6 are flow diagrams of methods for heart sound identification in accordance with one or more embodiments of the invention. In one or more embodiments of the invention, one or more of the steps shown in FIGS. 3-6 may be omitted, repeated, performed in parallel, and/or performed in a different order. Accordingly, the specific arrangement of steps shown in FIGS. 3-6 should not be construed as limiting the scope of the invention. Furthermore, in order to simply the flow diagrams, some error checking steps, storage steps, etc. may not be explicitly shown. However, one of ordinary skill in the art will understand that such steps may be included.

As shown in FIG. 3, initially an audio signal of heart sounds is received and normalized to increase the amplitude of the audio waveform to the maximum level (300). The audio signal may be normalized by locating the sample with the highest peak among the samples in the audio stream and then dividing each sample by the sample with highest peak. In some embodiments of the invention, the audio signal is of sufficient length to contain at least two consecutive S₁ peaks. In one or more embodiments of the invention, the audio signal is of sufficient length to contain at least three cardiac cycles.

Subsequently, the initial S₁ peak in the audio signal is identified within a search window beginning at the start of the audio signal (302). The length of this search window is an important factor in detecting S₁ and S₂ locations. Normal heart rate in healthy adults is usually between 60-100 BPMs. However, heart rates for newborns and children under the age of one can range from 100-180 BPMs for newborns and children under the age of one. If the window length is too small, the first S₂ peak in the audio signal may be identified as the subsequent S₁ peak (i.e., the S₁ peak at the beginning of the next cardiac cycle). If the window length is too large, the subsequent S₁ peak may not be found if the heart rate is at the higher end of the heart rate range. In one or more embodiments of the invention, two window lengths are used, a large window length and a small window length. The large window length, which is also the default window length, is used initially, and, as is explained in more detail below, if the use of this large window length fails to appropriately locate S₁ and S₂ peaks, the search window is decreased to the small window length and the audio signal is processed again using the smaller search window. Further, as is described in more detail below, a hop length (i.e., the distance to the starting location of the next search window) is decreased. In one or more embodiments of the invention, the large window length is 200 ms and the small window length is 100 ms.

The initial S₁ peak may be identified by finding a maximum value, i.e., the amplitude of the highest peak, and a minimum value, i.e., the amplitude of the lowest peak, within the search window. If the difference between the maximum value and the minimum value is greater than a predetermined amount, the highest peak may be the initial S₁ peak. If the difference between the maximum value and the minimum value is less than or equal to the predetermined amount, then the length of the search window is increased by a predetermined number of milliseconds and a new maximum value and minimum value are found. In one or more embodiments of the invention, this predetermined amount is 0.8 and the predetermined number of milliseconds is 50 ms.

The process of increasing the search window length and finding a new maximum value and minimum value is repeated until either a maximum value and a minimum value are found for which the difference is greater than the predetermined amount or a maximum length of the search window is reached. In one or more embodiments of the invention, this maximum length is 1200 ms. If the maximum length of the search window is reached without finding an acceptable maximum value and minimum value, then the maximum value within the maximum search window length is selected as a possible initial S₁ peak if the maximum value is greater than a predetermined amount. If this maximum value is not greater than the predetermined amount, an error is indicated and processing of the audio signal terminates. In one or more embodiments of the invention, this predetermined amount is 0.25.

Once a peak that may be the initial S₁ peak is located, this candidate peak is checked using time domain kurtosis to see if it may be a murmur peak. As one of ordinary skill in the art would know, an S₁ (or an S₂) may peak earlier than a murmur. In the methods described herein, this known early occurrence is exploited to distinguish an S₁ peak (or S₂ peak) from a later occurring murmur peak. Specifically, time domain kurtosis (i.e., kurtosis of the signal as it varies in the time domain) is used to distinguish an S₁ peak (or S₂ peak) from a murmur peak. Three kurtosis values are calculated in the time domain: a kurtosis (K) of the segment of the audio signal that is a predetermined number of milliseconds on either side of the candidate peak, a kurtosis (K₁) of segment that is the predetermined number of milliseconds before the candidate peak, and a kurtosis (K₂) of the segment that is the predetermined number of milliseconds after the candidate peak. In one or more embodiments of the invention, the predetermined number of milliseconds is 100. K is usually higher for an S₁ peak (or an S₂ peak) than for a murmur peak. Also, the difference between K₁ and K₂ for an S₁ peak (or an S₂ peak) is much larger than for a murmur peak. Accordingly, if K is greater than a predetermined value, V, or if the absolute difference between K₁ and K₂ is greater than a predetermined value, V₂, then the candidate peak is not a murmur. Otherwise, the candidate peak is a murmur. In one or more embodiments of the invention, V is 4.0 and V₂ is 6.0.

If the candidate peak is found to be a murmur peak, the search for the initial S₁ peak is continued as described above with an increased search window length. The location of this murmur peak may also be stored for later use in providing additional diagnostic information to identify the murmur. If the candidate peak is not found to be a murmur peak, then it is identified as the initial S₁ peak.

After identifying the initial S₁ peak, the search window is moved by a sufficient number of milliseconds, i.e., a hop length, to a location before the subsequent S₁ peak (i.e., the S₁ peak at the beginning of the next cardiac cycle) (304). More specifically, the beginning of the search window is moved to a location that is a hop length away from the initial S₁ peak. For purposes of locating the first S₁ peak after the initial S₁ peak, the length of this search window may be the same as the initial length of the search window used in identifying the initial S₁ peak, i.e., either the large window length or the small window length. In one or more embodiments of the invention, the hop length is 400 ms if the large window length is used and 200 ms if the small window length is used.

Referring again to FIG. 3, the subsequent S₁ peak is then identified within the relocated search window (304). The subsequent S₁ peak may be identified by finding the maximum value, i.e., the amplitude of the highest peak, within the search window. If the difference between the amplitude of the highest peak and the amplitude of the previous S₁ peak is within tolerance, then the highest peak may be the subsequent S₁ peak. In one or more embodiments of the invention, the difference between the amplitudes is within tolerance if the difference is less than 0.2. If the difference between the amplitudes is not within tolerance, then the length of the search is increased by a predetermined amount and the maximum value of the longer search window is found and compared to the amplitude of the previous S₁ peak. In one or more embodiments of the invention, this predetermined amount is 50 ms. The process of increasing the length of the search window and finding maximums is repeated until either an acceptable peak is found or the length of the search window reaches a maximum length. In one or more embodiments of the invention, this maximum length is 700 ms. If the length of the search window reaches this maximum length without an acceptable peak being located, the tolerance is increased by a predetermined amount and the search window is returned to its initial length. In one or more embodiments of the invention, this predetermined amount is 0.02 ms. The above described search for an acceptable peak is then repeated until either an acceptable peak is found or the tolerance reaches a maximum tolerance. In one or more embodiments of the invention, the maximum tolerance is 0.3.

If the maximum tolerance is reached without finding an acceptable peak, the maximum search window length is increased by a predetermined amount, the tolerance is returned to its initial value, and the above described search for an acceptable peak is repeated until either an acceptable peak is found or the maximum search window length reaches a predetermined length limit. In one or more embodiments of the invention, this predetermined amount is 100 ms and the predetermined length limit is 1200 ms.

If the predetermined length limit is reached without finding an acceptable peak, the maximum value, i.e., the amplitude of the highest peak, within the search window with a length of the predetermined length limit is found. If this maximum value is greater than a predetermined percentage of the amplitude of the previous S₁ peak, then this highest peak may be the subsequent S₁ peak. In one or more embodiments of the invention, this predetermined percentage is thirty-three percent. In some instances, a peak that is much smaller than the previous S₁ peak may be the subsequent S₁ peak. For example, if a ventricular septal defect is present, the subsequent S₁ peak can be much smaller than the previous S₁ peak. Also, improper recording or a change in auscultation location (i.e., where the stethoscope is placed on the chest) can cause variations in the amplitudes of S₁ peaks. If this highest peak does not have sufficient amplitude, then if a murmur peak was found while identifying the previous S₁ peak, the murmur peak is identified as the subsequent S₁ peak. If no murmur peak was found, an error is indicated and the processing of the audio signal terminates.

Once a peak that may be the subsequent S₁ peak is located, this candidate peak is checked using time domain kurtosis as described above to see if it may be a murmur peak. If the candidate peak is found to be a murmur peak, the search for the subsequent S₁ peak is continued as described above with an increased search window length. The location of this murmur peak may also be stored for later use in providing additional diagnostic information to identify the murmur. If the candidate peak is not found to be a murmur peak, then it is identified as the subsequent S₁ peak.

Referring again to FIG. 3, once the subsequent S₁ peak is identified, an S₂ peak between the previous S₁ peak and the subsequent S₁ peak is identified (306). The S₂ peak may be identified by finding a maximum value, i.e., the amplitude of the highest peak, between the previous S₁ peak and the subsequent S₁ peak. More specifically, the maximum value is found for a segment that begins at a location determined by the sum of the location of the previous S₁ peak and a predetermined duration of an S₁ peak and ends at a location determined by the difference between the location of the subsequent S₁ peak and a predetermined duration of an S₂ peak. In one or more embodiments of the invention, the predetermined duration of an S₁ peak is 150 ms and the predetermined duration of an S₂ peak is 120 ms. If this maximum value is greater than a predetermined percentage of the difference between the maximum value and minimum value found when identifying the initial S₁ peak, then this highest peak may be the S₂ peak. In one or more embodiments of the invention, this predetermined percentage is 12.5 percent. Further, in one or more embodiments of the invention, if the maximum value meets this criterion, this maximum value is checked using time domain kurtosis as described above to see if it may be a murmur peak.

If the maximum value does not meet the criterion (or in embodiments in which the murmur check is performed, the maximum value is found to be a murmur peak, then a maximum value, i.e., the amplitude of the highest peak, is found for a segment that begins at the same location as above and ends a location determined by the sum of the location of the previous S₁ peak and a predetermined percentage of the length of the search window in which the subsequent S₁ peak was found. In one or more embodiments of the invention, this predetermined percentage is seventy-five percent. If this maximum value is greater than a predetermined percentage of the difference between the maximum value and minimum value found when identifying the initial S₁ peak, then this highest peak may be the S₂ peak. In one or more embodiments of the invention, this predetermined percentage is 12.5 percent.

If the maximum value does not meet this criterion, then if the previous S₁ peak is the initial S₁ peak, the previous S₁ peak is actually an S₂ peak that occurred at the beginning of the audio signal. Although not specifically shown in FIG. 3, the subsequent S₁ peak is accepted as the initial S₁ peak (i.e., the S₁ peak at the beginning of the initial full cardiac cycle in the audio signal) and the method loops back to (304) to repeat the identification of the subsequent S₁ peak and the S₂ peak.

If the previous S₁ peak is not the initial S₁ peak, then the peak at the location determined by the sum of the location of the previous S₁ peak and an average distance between an S₁ peak and an S₂ peak may be the S₂ peak. In one or more embodiments of the invention, the default average distance between an S₁ peak and an S₂ peak is 350 ms. As is explained in more detail below, the average distance may be adjusted as S₁ and S₂ peaks are located.

Once a peak that may be the S₂ peak is located, this candidate S₂ peak is checked to see if it is an S₃ peak or an opening snap peak. This check may be performed as follows. First, the maximum value, i.e., the amplitude of the highest peak, is found for a segment that begins at a location determined by the sum of the location of the previous S₁ peak and the predetermined duration of an S₁ peak and ends at a location determined by the difference between the location of the candidate S₂ peak and a predetermined percentage of the predetermined duration of an S₂ peak. In one or more embodiments of the invention, this predetermined percentage is thirty-three percent. If this maximum value is less than a predetermined percentage of the amplitude of the candidate S₂ peak, then the candidate S₂ peak is not an S₃ peak or an opening snap peak and is identified as the S₂ peak. In one or more embodiments of the invention, this predetermined percentage is fifty percent.

If the maximum value meets the amplitude criteria, the new peak is checked using frequency domain kurtosis (i.e., kurtosis of the signal as it varies in the frequency domain) to determine whether it is a late systolic murmur peak. More specifically, kurtosis of the Fourier transform magnitude is used to determine if the new peak is due to a murmur. The magnitude of the Fourier transform of a segment beginning at the location of the candidate S₂ peak is computed and the associated kurtosis measure, G1, is found. Similarly, the magnitude of the Fourier transform of a segment beginning at the location of the new peak is computed and the associated kurtosis measure, G2, is found. In one or more embodiments of the invention, the length of the segments is the nearest power of two to the length in samples that equals 50 ms of time. For example, the length is 512 if the sampling frequency is 11025 Hz and 256 if the sampling frequency is 4000 Hz. If the absolute difference between the geometric mean of G1 and G2 and the arithmetic mean of G1 and G2 is greater than a predetermined value and if G1 is greater than G2, then the new peak is identified as a possible murmur peak. In one or more embodiments of the invention, this predetermined value is 3.5.

If the new peak is not a found to be a murmur peak, then it is identified as the S₂ peak and the candidate S₂ peak is identified as a possible S₃ peak or opening snap peak. In one or more embodiments of the invention, the location of the possible S₃/opening snap peak may be stored for later use in providing additional diagnostic information regarding the presence of S₃/opening snap peaks in the heart sounds. If the new peak is a possible late systolic murmur, then the candidate S₂ peak is identified as the S₂ peak. In one or more embodiments of the invention, the location of the late systolic murmur peak may be stored for later use in providing additional diagnostic information to identify the murmur.

Once the S₂ peak is identified, a check is then made to verify that the distance between the previous S₁ peak and the S₂ peak is smaller than the distance between the S₂ peak and the subsequent S₁ peak (308). If this distance check fails, then different actions are taken depending on whether or not S₁ and S₂ peaks are being identified for the initial cardiac cycle or a subsequent cardiac cycle (320). If the initial S₁ and S₂ peaks of the first full cardiac cycle in the audio signal are being identified, then the previous S₁ peak is actually an S₂ peak that occurred at the beginning of the audio signal. The beginning of the search window is moved to a location that is a hop length away from the subsequent S₁ peak. The subsequent S₁ peak is identified as the initial/previous S₁ peak (i.e., the S₁ peak at the beginning of the initial cardiac cycle in the audio signal) (322) and the method loops back to (304) to repeat the identification of the subsequent S₁ peak and the S₂ peak.

If the initial S₁ and S₂ peaks are not being identified (320), then a check is made to determine if there are peaks between the previous S₁ peak and the S₂ peak that are not murmurs (324). More specifically, a check is made to determine if there is a valid S₁ peak and a valid S₂ peak between the identified previous S₁ peak and the identified S₂ peak. If valid S₁ and S₂ peaks are found, the length of the search window is too large. The processing of the audio signal is restarted (302) using the small window length and a smaller hop length. In one or more embodiments of the invention, the smaller hop length is one half of the hop length used with the large window length. Further, the average distances between peaks (discussed below) and the expected durations of S₁ and S₂ are also reset to smaller initial values. In one or more embodiments of the invention, the smaller initial values are one half of the initial values used with the large window length.

If valid S₁ and S₂ peaks are not found, then a check is made to determine if the distance between the previous S₁ peak and the subsequent S₁ peak is acceptable (326). This check is made because it is possible for an S₂ peak to be selected as the subsequent S₁ peak if the next S₁ is a small peak. In one or more embodiments of the invention, a check is made to determine if the distance between the previous S₁ peak and the subsequent S₁ peak is within a predetermined percentage of an average distance between S₁ peaks. In one or more embodiments of the invention, the default average distance between S₁ peaks is 800 ms and, as is explained in more detail below, the average distance is adjusted as S₁ peaks are located in the audio signal. Further, in one or more embodiments of the invention, the predetermined percentage is twenty percent.

If the distance is acceptable, then no change is made to the identified subsequent S₁ peak and the method continues with timing based error correction (312) as described below. If the distance is not acceptable, a new maximum value found within an acceptable distance of the previous S₁ peak, this new peak is identified as the subsequent S₁ peak (328), and the method continues with timing based error correction (312) as described below. In one or more embodiments of the invention, the new maximum value is found in the segment beginning at a location determined by the sum of the location of the previous S₁ peak and the difference between the average distance between S₁ peaks and a predetermined percentage of the average distance (i.e., location+average distance−percentage of average distance) and ending at a location determined by the sum of the location of the previous S₁ peak, the average distance between S₁ peaks, and the predetermined percentage of the average distance (i.e., location+average distance+percentage of average distance). In one or more embodiments of the invention, the predetermined percentage is ten percent.

The check to determine if there is a valid S₁ peak and a valid S₂ peak between the identified previous S₁ peak and the identified S₂ peak may be done as follows. The two largest peaks, peak1 and peak2, between the previous S₁ peak and the S₂ peak are located, where peak1 refers to the peak closer to the S₂ peak and peak2 refers to the peak closest to the previous S₁ peak. If the difference in amplitude between the previous S₁ peak and peak1 is greater than a predetermined amount or the difference in amplitude between the S₂ peak and peak2 is greater than the predetermined amount, then there are no valid peaks between the previous S₁ peak and the S₂ peak and no other checking needs to be performed. In one or more embodiments of the invention, this predetermined amount is 0.3. Otherwise, if the distance between peak1 and peak2 is smaller than a predetermined percentage of the distance between the previous S₁ peak and the S₂ peak, then there are no valid peaks between the previous S₁ peak and the S₂ peak and no further checking needs to be performed. In one or more embodiments of the invention, this predetermined percentage is twenty-five percent.

If the distance between peak1 and peak2 does not meet this criterion, then a check is made to determine if peak1 and peak2 are murmur peaks. This check is made using time domain kurtosis. More specifically, the kurtosis, h1, of the segment beginning and ending a predetermined number of milliseconds on either side of the location of peak 1 is computed and the kurtosis, h2, of the segment beginning and ending the predetermined number of milliseconds on either side of the location of peak2 is computed. In one or more embodiments of the invention, this predetermined number of milliseconds is 75 ms. If the absolute value of the ratio of the maximum of h1 and h2 and the minimum of h1 and h2 is greater than a predetermined value, then peak1 and peak2 are murmur peaks and there are no valid peaks between the previous S₁ peak and the S₂ peak. In one or more embodiments of the invention, this predetermined value is 1.2. If the absolute value does not meet this criterion, then there are valid peaks between the previous S₁ peak and the S₂ peak.

Referring again to FIG. 3 and returning to the previously mentioned distance check (308), if the distance check is successful, then different actions are taken depending on whether or not the initial S₁ and S₂ peaks are being identified (310). If the initial S₁ and S₂ peaks are not being identified, then timing based error correction is performed to correct the S₂ peak and/or the subsequent S₁ peak, if correction is needed (312). In general, timing based error correction helps ensure that appropriate S₁ and S₂ peaks are identified when pathological conditions such as continuous murmur, aortic regurgitation, aortic stenosis, and ejection click are present. Such pathological conditions can cause the wrong peaks to be selected in some circumstances. Thus, timing based error correction is performed to further ensure that the appropriate peaks have been picked for the S₂ peak and the subsequent S₁ peak.

Timing based error correction compares certain distances (i.e., amount of time elapsed) between the previous S₁ peak, the S₂ peak, the subsequent S₁ peak, and/or the previous S₂ peak (i.e., the S₂ peak identified for the previous cardiac cycle) against expected distances between such peaks. If an actual distance exceeds an expected distance by more than a predetermined threshold, an attempt is made to locate a peak that is within the expected distance. If such a peak is located, it is identified as the subsequent S₁ peak or the S₂ peak, depending on which distance is being checked. Further, if changes are made to either the subsequent S₁ peak or the S₂ peak during the correction process, information regarding the changes may be stored for later use in providing additional diagnostic information related to the identification of murmurs. For example, if any subsequent S₁ peak is corrected, this correction may be indicative of aortic stenosis. In addition, if S₂ peaks are corrected, aortic regurgitation may be present.

In one or more embodiments of the invention, timing based error correction is performed as follows. Initially, the distance between the previous S₁ peak and the subsequent S₁ peak is checked. If the distance is not within a predetermined percentage of the average distance between two S₁ peaks, then a search is performed for a nearby peak that meets this criterion and could possibly be the “real” subsequent S₁ peak. In one or more embodiments of the invention, the predetermined percentage is twenty percent. Further, in one or more embodiments of the invention, the average distance between two S₁ peaks is initially set to 800 ms. This average distance is updated using the actual distance between the previous S₁ peak and the subsequent S₁ peak after the time based error correction process is complete.

To locate a nearby peak, the highest peak in the segment that begins at the location determined by the sum of the location of the previous S₁ peak and the average distance between S₁ peaks less the predetermined percentage of the average distance (location+average distance−percentage of average distance) and ends at the location determined by the sum of the location of the previous S₁ peak, the average distance, and the predetermined percentage of the average distance (location+average distance+percentage of average distance). If the amplitude of the new peak is greater than a predetermined percentage of the previous S₁ peak, then the new peak is identified as the subsequent S₁ peak. Otherwise, the subsequent S₁ peak is not changed. In one or more embodiments of the invention, the predetermined percentage is thirty-three percent.

Next, the distance between the previous S₂ peak and the S₂ peak is checked. If the distance is not within a predetermined percentage of the average distance between two S₂ peaks, then a search is performed for a nearby peak that meets this criterion and could possibly be the “real” S₂ peak. In one or more embodiments of the invention, this predetermined percentage is twenty percent. Further, in one or more embodiments of the invention, the average distance between two S₂ peaks is initially set to 800 ms. This average distance is updated using the actual distance between the previous S₂ peak and the S₂ peak after the time based error correction process is complete.

To locate a nearby peak, the highest peak in the segment that begins at the location determined by the sum of the location of the previous S₂ peak and the average distance between S₂ peaks less the predetermined percentage of the average distance (location+average distance−percentage of average distance) and ends at the location determined by the sum of the location of the previous S₂ peak, the average distance, and the predetermined percentage of the average distance (location+average distance+percentage of average distance). If the amplitude of the new peak is greater than a predetermined percentage of the previous S₂ peak, then the new peak is identified as the S₂ peak. Otherwise, the S₂ peak is not changed. In one or more embodiments of the invention, the predetermined percentage is thirty-three percent.

Next, the distance between the previous S₁ peak and the S₂ peak is checked. If the distance is not within a predetermined percentage of the average distance between an S₁ peak and an S₂ peak in the same cardiac cycle, then a search is performed for a nearby peak that meets this criterion and could possibly be the “real” S₂ peak. In one or more embodiments of the invention, the predetermined percentage is twenty percent. Further, in one or more embodiments of the invention, the average distance between an S₁ peak and an S₂ peak in the same cardiac cycle is initially set to 350 ms. This average distance is updated using the actual distance between the previous S₁ peak and the S₂ peak after the time based error correction process is complete.

To locate a nearby peak, the highest peak in the segment that begins at the location determined by the sum of the location of the previous S₁ peak and the average distance between an S₁ peak and an S₂ peak in the same cardiac cycle less the predetermined percentage of the average distance (location+average distance−percentage of average distance) and ends at the location determined by the sum of the location of the previous S₁ peak, the average distance, and the predetermined percentage of the average distance (location+average distance+percentage of average distance). If the amplitude of the new peak is greater than a predetermined percentage of the previous S₂ peak, then the new peak is identified as the S₂ peak. Otherwise, the S₂ peak is not changed. In one or more embodiments of the invention, the predetermined percentage is thirty-three percent.

Finally, the distance between the S₂ peak and the subsequent S₁ peak is checked. If the distance is not within a predetermined percentage of the average distance between an S₂ peak in one cardiac cycle and the S₁ peak of the next cardiac cycle, then a search is performed for a nearby peak that meets this criterion and could possibly be the “real” subsequent S₁ peak. In one or more embodiments of the invention, the predetermined percentage is twenty percent. Further, in one or more embodiments of the invention, the average distance between an S₂ peak in one cardiac cycle and the S₁ peak of the next cardiac cycle is initially set to 450 ms. This average distance is updated using the actual distance between the S₂ peak and the subsequent S₁ peak after the time based error correction process is complete.

To locate a nearby peak, the highest peak in the segment that begins at the location determined by the sum of the location of the S₂ peak and the average distance between an S₂ peak in one cardiac cycle and the S₁ peak of the next cardiac cycle less the predetermined percentage of the average distance (location+average distance−percentage of average distance) and ends at the location determined by the sum of the location of the S₂ peak, the average distance, and the predetermined percentage of the average distance (location+average distance+percentage of average distance). If the amplitude of the new peak is greater than a predetermined percentage of the subsequent S₁ peak, then the new peak is identified as the subsequent S₁ peak. Otherwise, the subsequent S₁ peak is not changed. In one or more embodiments of the invention, the predetermined percentage is thirty-three percent.

Referring again to FIG. 3, after timing based error correction is performed (312) or if the initial S₁ and S₂ peaks are being identified (310), a check is made to determine if there are peaks between the previous S₁ peak and the S₂ peak that are not murmurs (314). More specifically, a check is made to determine if there is a valid S₁ peak and a valid S₂ peak between the identified previous S₁ peak and the identified S₂ peak. This check may be performed as described above. If valid S₁ and S₂ peaks are found, the length of the search window is too large. The processing of the audio signal is restarted (302) using the small window length and a smaller hop length. In one or more embodiments of the invention, the smaller hop length is one half of the hop length used with the large window length. Further, the previously mentioned average distances between peaks are also reset to smaller initial values. In one or more embodiments of the invention, the smaller initial values are one half of the initial values used with the large window length.

If valid S₁ and S₂ peaks are not found between the previous S₁ peak and the S₂ peak, then if the end of the audio signal has not been reached (316), the next S₂ peak and S₁ peak in the audio signal are located. The beginning of the search window is moved to a location that is a hop length away from the subsequent S₁ peak. The method then loops back to identify the next S₁ peak and S₂ peak in the audio signal (304) as described above. Note that the subsequent S₁ peak becomes the previous S₁ peak in the new iteration.

If the end of the audio signal has been reached (316), then the heart rate and/or other diagnostic information may be calculated and displayed (318) in a PCG. In addition, the locations of the S1 and S2 peaks may demarcated in the PCG using symbols, colors, and/or any other suitable demarcation scheme. Further, in one or more embodiments of the invention, the heart rate and/or other diagnostic information may also be calculated and displayed along with the PCG as the audio signal is being analyzed rather than waiting until end of the signal is reached.

The heart rate may be determined based on the number of S₁ peaks located in the audio signal and the sampling frequency of the signal. More specifically, if Lₛ is the number of S₁ peaks, Fₛ is the sampling frequency of the audio signal, x is the location of the first S₁ peak in the signal, and y is the location of the last S₁ peak in the signal, then the heart rate is equal to ((Lₛ−1)*60*Fₛ)/(y−x) BPM.

The other diagnostic information that may be calculated and displayed depends upon what information may have been stored during the analysis of the audio signal. For example, the types of murmurs are generally indicated by where in the cardiac cycle the murmur is located. For example, a diastolic murmur sound occurs after the S₂ sound, a systolic murmur sound occurs between the S₁ sound and the S₂ sound, with an early systolic murmur sound occurring close to the S₁ sound and a late systolic murmur sound occurring close to the S₂ sound. If the locations of potential murmurs as detected by the previously described kurtosis computations are stored, this information can be used in conjunction with S₁ and S₂ locations to help determine what type of murmur is present. Information saved during timing based error correction regarding correction of S₁ and S₂ peaks may also be used to provide diagnostic information. As previously mentioned, if any S₁ peak is corrected by the timing based error correction, aortic stenosis may be indicated. Further, if S₂ peaks are corrected by timing based error correction, aortic regurgitation may be indicated. In addition, once a murmur peak is located, it is possible to provide the time duration of the murmur and information regarding the intensity and frequency content of the murmur.

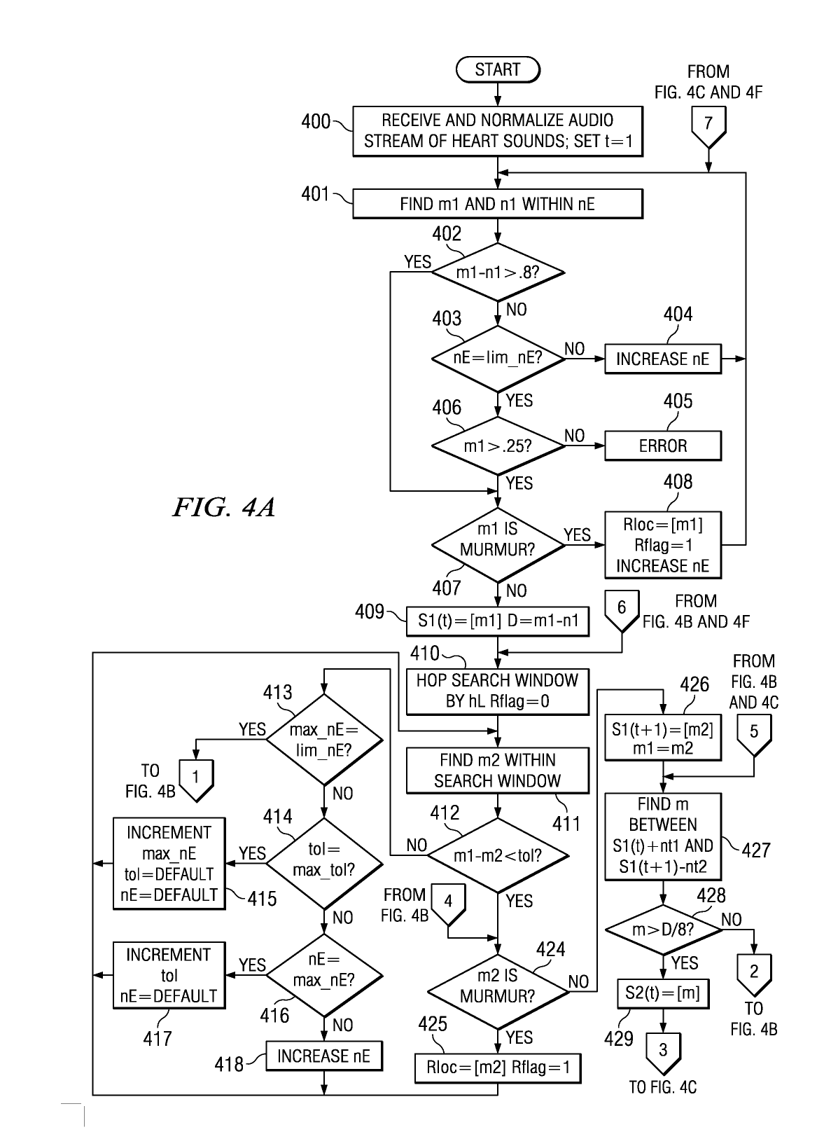

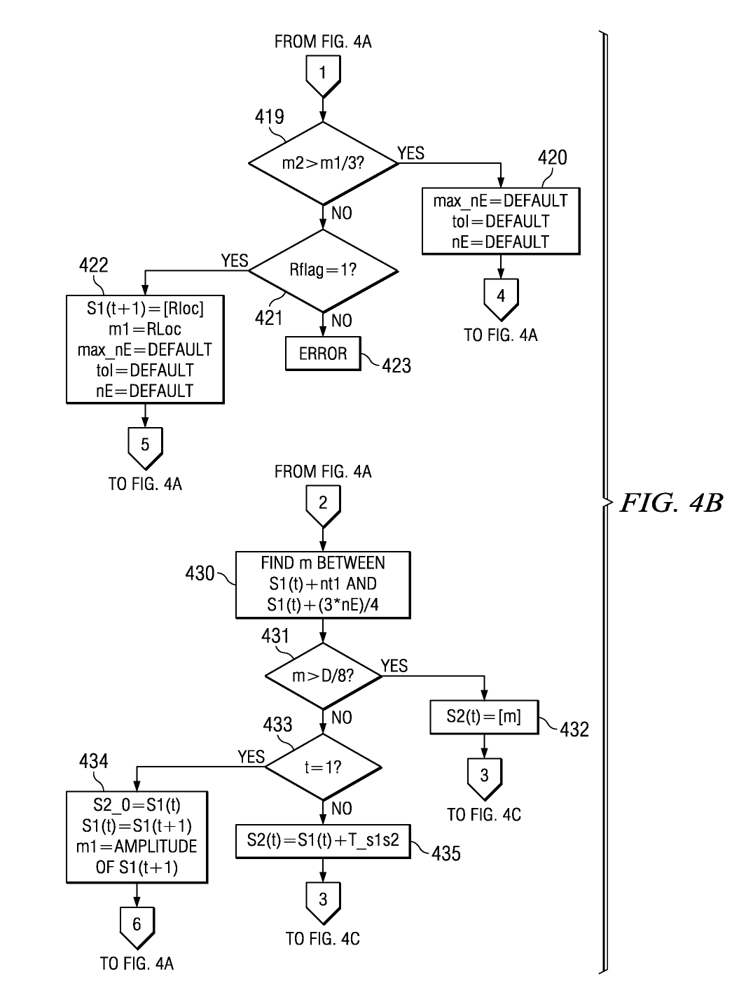

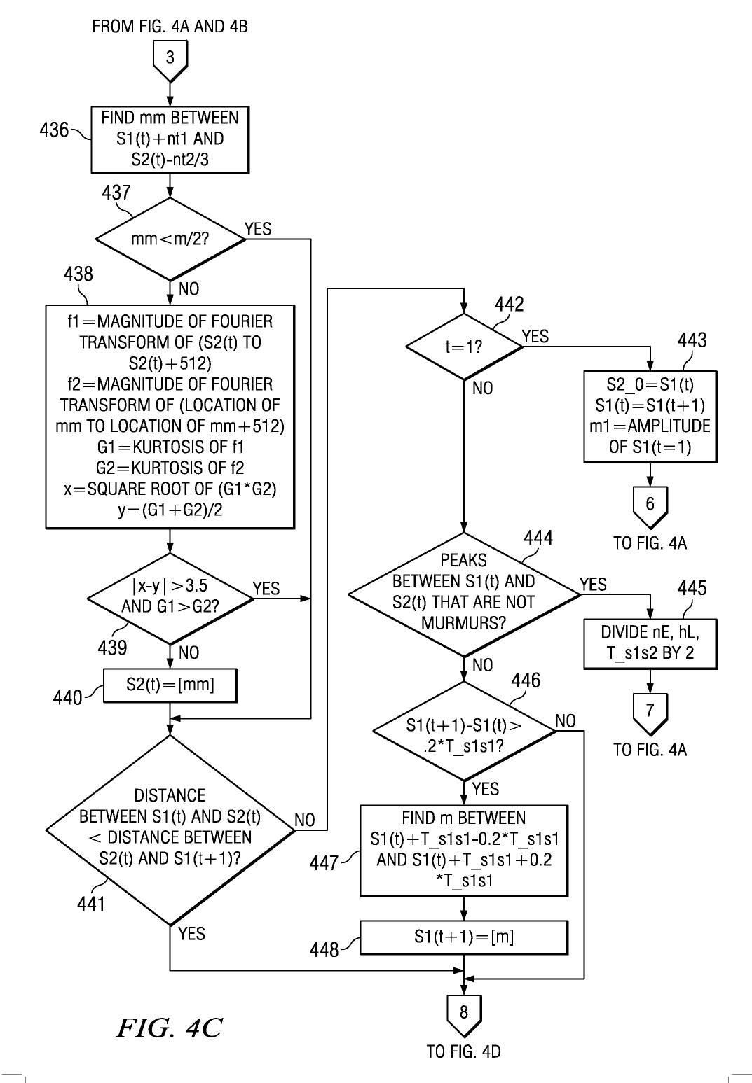

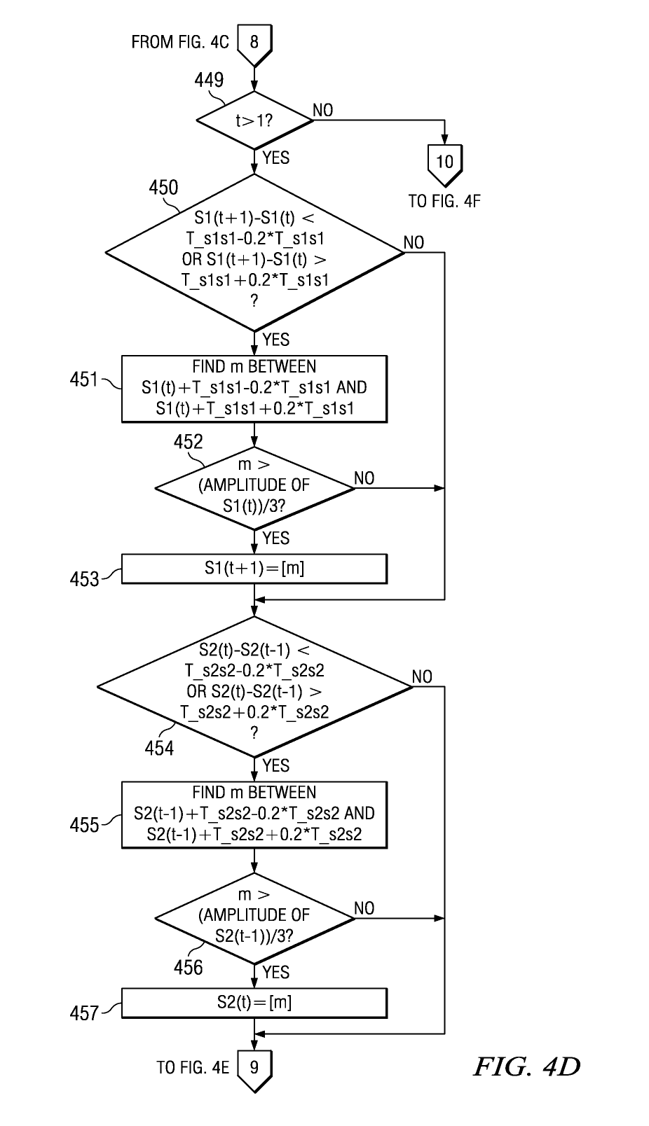

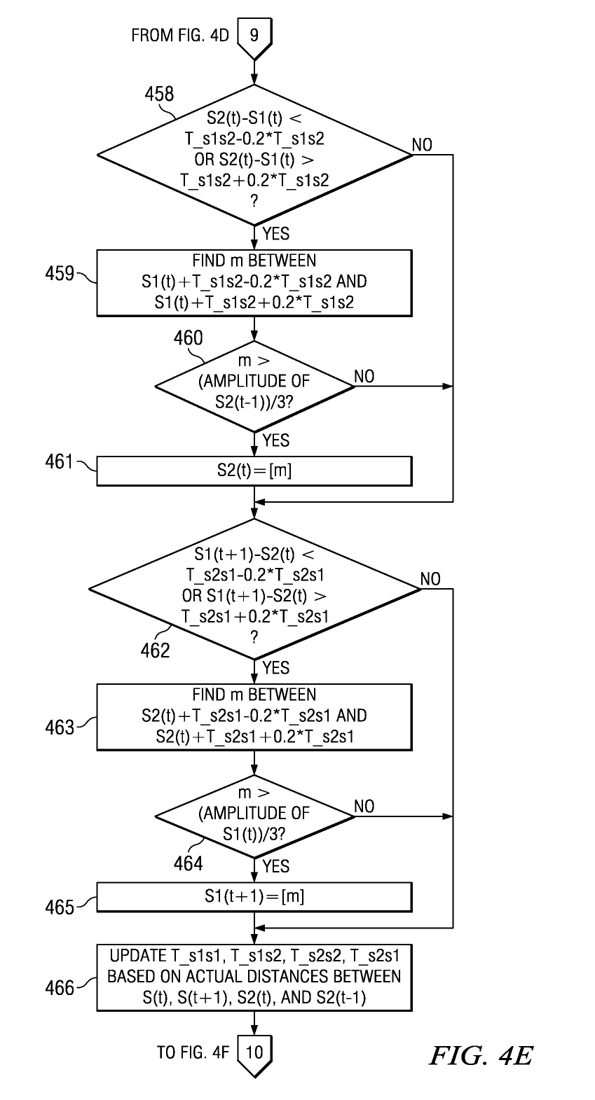

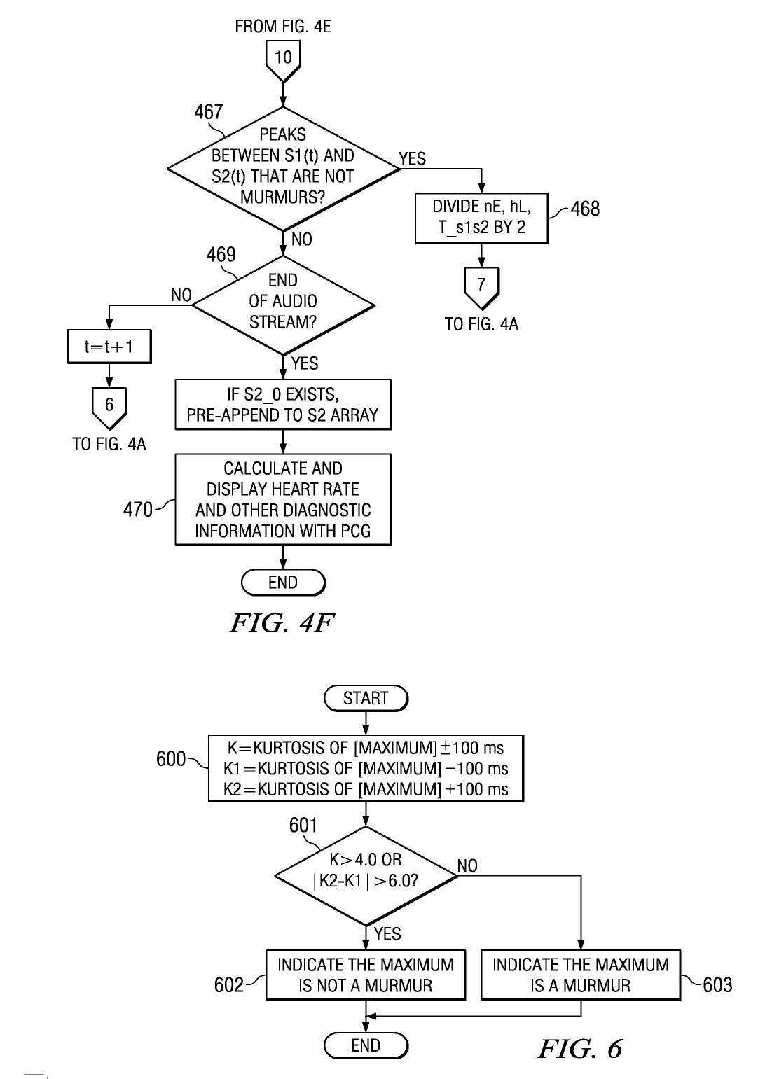

Turning now to FIG. 4, Table 1 defines the symbols used in the flow graph. In addition, in the flowgraph, [symbol] means “location of.” For example, [m1] means location of m1. Many of the values and defaults presented in this table and the numbers specified in FIG. 4 are empirically derived from implementing embodiments of the method and executing the implementations with sample audio streams of heart sounds, both normal heart sounds and heart sounds including a wide variety of pathological conditions. The particular values, defaults, and numbers were found to provide optimal performance in view of all of the sample audio streams. However, variations from these values, defaults, and numbers may be used without departing from the scope of the invention.

TABLE 1

| Symbol | Definition |
|---|---|
| nE | Search window length (default = 200 ms) |
| hL | Hop length; the distance to move the search window from the current S1 |
| | to the location to start the search for the next S1 (default = 400 ms) |
| tol | Tolerance, i.e., allowable difference in amplitude, between consecutive |
| | S1 peaks (default = 0.2) |
| max_tol | The maximum value, 0.3, to which tol may be incremented |
| max_nE | The maximum value, 700 ms, to which nE may be incremented before tol |
| | is incremented |
| lim_nE | The absolute maximum value, 1200 ms, the which nE may be |
| | incremented |
| S1 | The array storing locations of S1 peaks in the audio signal |
| S2 | The array storing locations of S2 peaks in the audio signal |
| t | Index variable into S1 and S2, that store locations of S1 and S2 peaks |
| S2_0 | Location of an S₂ peak at the beginning of the signal that occurred before |
| | the first S₁ peak in the signal |
| nt1 | Duration of an S₁ heart sound (default = 150 ms) |
| nt2 | Duration of an S₂ heart sound (default = 120 ns) |
| D | Difference between the maximum and minimum values within the first |
| | search window |
| RFlag | Flag set to indicate a murmur |
| RLoc | Location of the murmur |
| T_s1s2 | Average distance between an S₁ peak and the following S₂ peak (default = 350 ms) |
| T_s1s1 | Average distance between two consecutive S₁ peaks (default = 800 ms) |
| T_s2s1 | Average distance between an S₂ peak and the next S₁ peak (default = 450 ms) |
| T_s2s2 | Average distance between two consecutive S₂ peaks (default = 800 ms) |
| m1, m2, m, | Maximum values, i.e., the amplitude of the highest peak in a segment of |
| mm, m3, | the audio signal |
| m4 | |
| n1 | Minimum value, i.e., the amplitude of the lowest peak in the search |
| | window in which the initial S₁ peak in the audio signal is found |

With the definitions provided in Table 1, the flow graph in FIG. 4 is easily understood by one of ordinary skill in the art without detailed explanation. Accordingly, additional explanation is provided only for certain portions of the flow graph.

Initially, an audio signal of heart sounds is received and normalized and t is set to 1 (400). A peak is then located within a search window that meets the criteria for being the initial S₁ peak (i.e., S1 (t)) in the signal (401-406). The located peak is then tested to see if it is a murmur peak (407). The test for a murmur peak is described below in reference to FIG. 6. If the peak is a murmur peak, the presence of the murmur peak is remembered (408) and another peak is located that meets the criteria for being the initial S₁ peak (401-406). This location process is repeated until a peak that meets the criteria and is not a murmur peak is located.

When the initial S₁ peak is located (409), the search window is moved (410), and a peak is located within the search window that meets the criteria for being the next S₁ peak (i.e., S1 (t+1)) in the audio signal is located (411-423). The located peak is then tested to see if it is a murmur peak (424). The test for a murmur peak is described below in reference to FIG. 6. If the peak is a murmur peak, the presence of the murmur peak is remembered (425) and another peak is located that meets the criteria for being the next S₁ peak (411-423). This location process is repeated until a peak that meets the criteria and is not a murmur peak is located.

When the next S₁ peak is located (426), a peak between the previous S₁ peak and the next S₁ peak is located that meets the criteria for being the S₂ peak (427-435). Once this candidate S₂ peak is located, the candidate S₂ peak is checked to see if it is actually an S₃ peak or a late systolic murmur peak (436-439). If the candidate S₂ peak is found to be an S₃ peak or a late systolic murmur peak, another peak is identified as the S₂ peak (440). Otherwise, the candidate S₂ peak is accepted as the S₂ peak, pending timing based error correction. The checking of the candidate S₂ peak to see if it is a late systolic murmur includes performing frequency domain kurtosis (438-439).

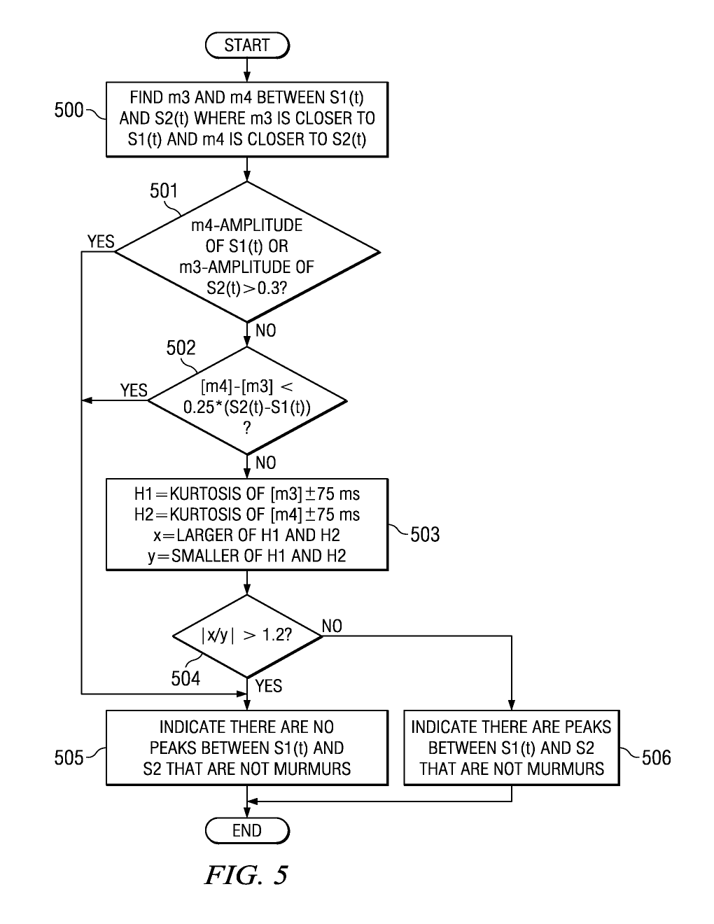

Once the S₂ peak is located, further checks are performed to ensure that the peaks located for the previous S₁ peak, the next S₁ peak, and the S₂ peak are actually the previous (or initial) S₁ peak, the next S₁ peak, and the S₂ peak (441-448). One of the checks that may be performed is a check to see if there are peaks between the previous S₁ peak and the S₂ peak that are not murmurs (444-445), i.e., that there are peaks between peaks selected as the previous S₁ peak and the S₂ peak that may also be S₁ and S₂ peaks. The check for non-murmur peaks is performed only if the distance between the previous S₁ peak and the S₂ peak is greater than the distance between the S₂ peak and the next S₁ peak. This check for non-murmur peaks is described below in reference to FIG. 5.

If the further checks are successfully completed, then if the first iteration of the S₁/S₂ location process has been completed (449) (i.e., the S₁ peak and S₂ peak for the first cardiac cycle in the audio stream have been located), timing based error correction is performed to further ensure that the peaks located for S₂ and the next S₁ are the correct peaks (450-465). As was previously discussed, timing based error correction uses various average distances between S₁ and or S₂ peaks to verify the current selections for the S₂ peak and the next S₁ peak. After timing based error correction is performed, the average distances are updated based on the locations of the S₁ and S₂ peaks located in the current iteration (466).

A final check is then made to ensure that the peaks located for previous S₁ peak and the S₂ peak are actually the previous (or initial) S₁ peak and the S₂ peak (441-448). This final check is a check to see if there are peaks between the previous S₁ peak and the S₂ peak that are not murmurs, i.e., that there are peaks between peaks selected as the previous S₁ peak and the S₂ peak that may also be S₁ and S₂ peaks (467-468). This check for non-murmur peaks is described below in reference to FIG. 5. If non-murmur peaks are found, then the identification process is restarted.

If non-murmur peaks are not found between the previous S₁ peak and the S₂ peak, and the end of the audio signal has not been reached (469), the method loops back to (410) to locate the next S₂ peak and the next S₁ peak in the audio signal. If the end of the audio signal has been reached, then the heart rate and other diagnostic information may be calculated and displayed in a PCG of the audio signal (470). In addition, the locations of the S₁ and S₂ peaks may demarcated in the PCG using symbols, colors, and/or any other suitable demarcation scheme.

FIG. 5 shows a flow diagram of a method for determining whether there are non-murmur peaks between two peaks that have been selected as the previous S₁ peak and the S₂ peak. First, two maximum values are found between the previous S₁ peak and the S₂ peak (500). If the difference between the amplitude of the maximum value closer to the S₂ peak and the amplitude of the previous S₁ peak is greater than 0.3 or the difference between the amplitude of the maximum value closer to the previous S₁ peak and the amplitude of the S₂ peak is greater than 0.3 (501), then there are no S₁/S₂ peaks between the previous S₁ peak and the S₂ peak that are not murmurs (505). Otherwise, if the absolute difference between locations of the two maximum values is less than twenty-five percent of the distance between the previous S₁ peak and the S₂ peak (502), again there are no peaks between the previous S₁ peak and the S₂ peak that are not murmurs (5054). Otherwise, frequency domain kurtosis is used to determine if the two maximum values are murmurs (503-504). If the maximum values are murmur peaks, then again there are no peaks between the previous S₁ peak and the S₂ peak that are not murmurs (505). Otherwise, there are peaks between S₁ and S₂ that are not murmurs (506).

FIG. 6 shows a flow diagram of a method for determining whether a peak that has been selected as a possible S₁ peak is a murmur peak. Initially, kurtosis in the time domain is computed for the segment 100 ms on either side of the location of the possible S1 peak (K), the segment 100 ms before the location of the possible S1 peak (K1), and the segment 100 ms after the possible S1 peak (600) (K2). If K is greater than 4.0 or the absolute difference between K1 and K2 is greater than 6.0 (601), then the possible S1 peak is not a murmur (602). Otherwise, the possible S1 peak is a murmur (603).

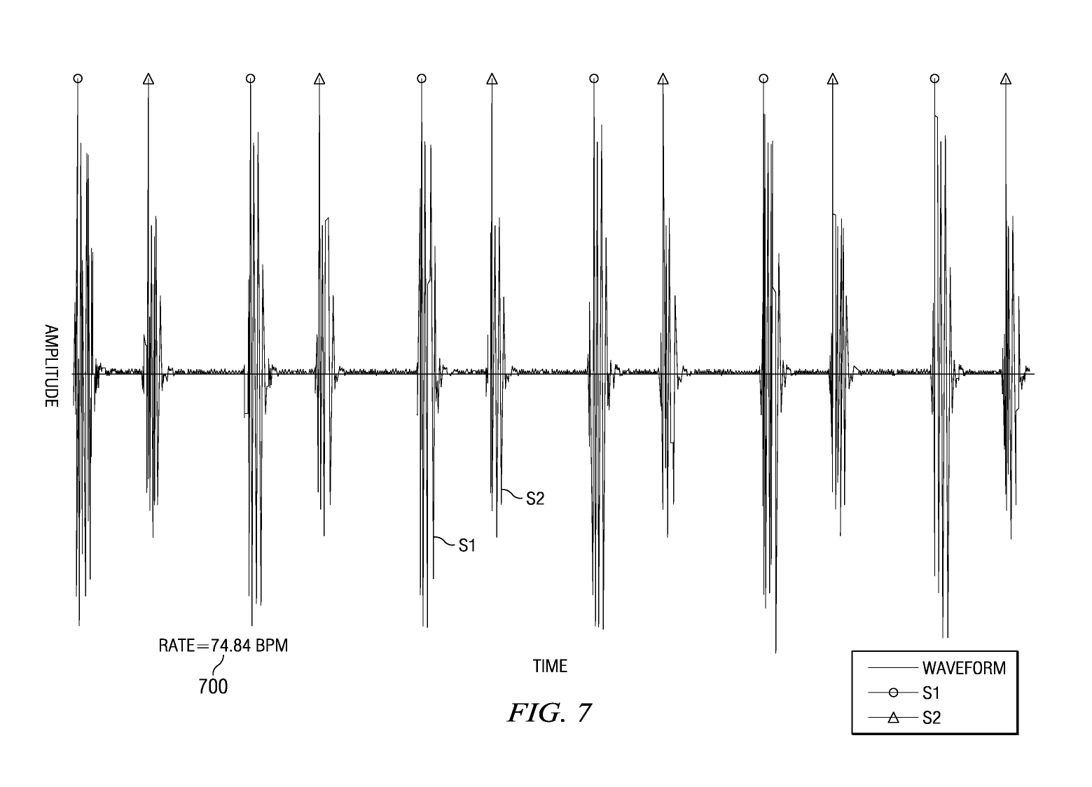

FIGS. 7-18 show example phonocardiograms (PCGs) of the results of applying an implementation of an embodiment of a method described herein to sample audio signals of heart sounds. In each of these PCGs, the heart rate resulting from the analysis of the signal is displayed, and each _{S1} peak and _{S2} peak identified in the analysis is labeled. For those heart sounds that included a cardiac abnormality, the cardiac abnormality is also identified.

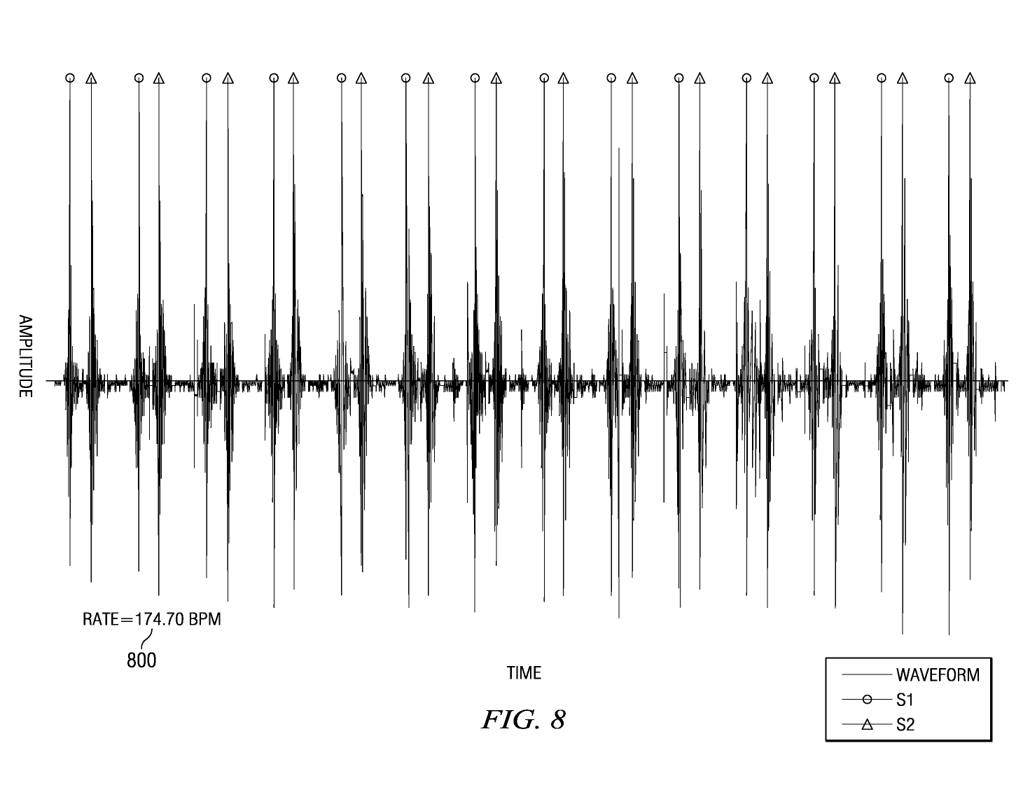

FIGS. 7 and 8 show PCGs of the results of analyzing audio signals with only normal heart sounds. The two figures illustrate that embodiments of the methods are robust for a wide range of heart rates. The heart rate (700) in the PCG of FIG. 7 is within the normal range for a healthy adult (i.e., 60-100 BPM) while the heart rate (800) in the PCG of FIG. 8 is at the high end of the normal range for a child under the age of one (i.e., 100-180 BPM).

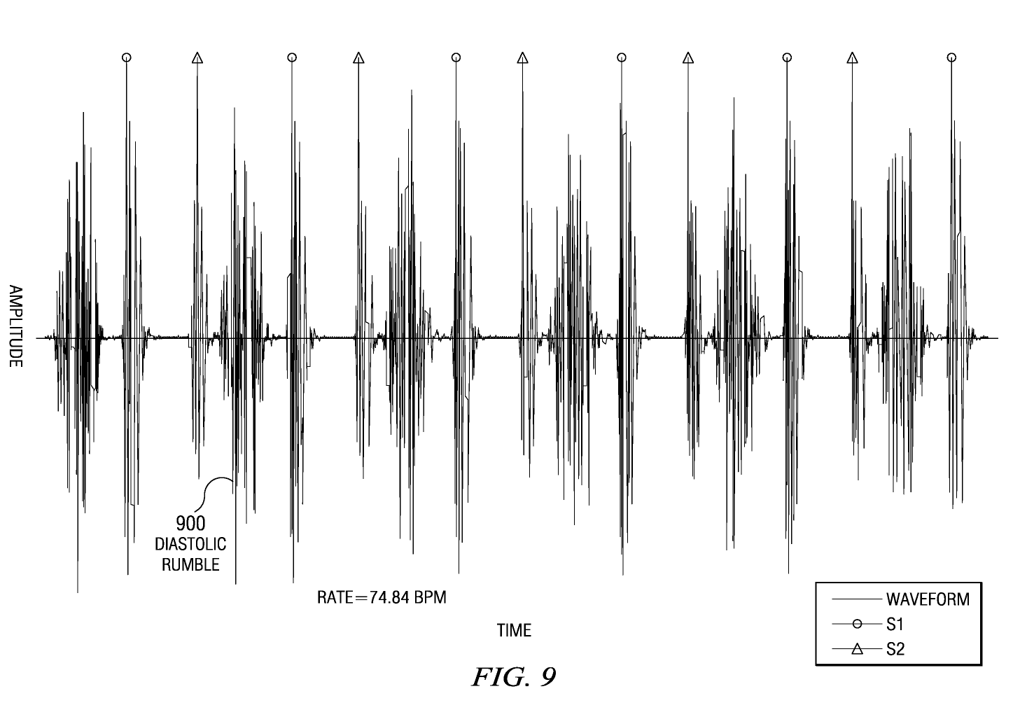

FIGS. 9-14 show PCGs of the results of analyzing audio signals with heart sounds that include various types of murmurs. These figures illustrate the ability of the methods to distinguish the primary heart sounds, S₁ and S₂, from heart sounds introduced by murmurs. FIG. 9 shows the result of analyzing an audio signal of heart sounds that include a diastolic rumble (900). The diastolic rumble sound occurs after the S₂ sound and its duration and intensity can vary from subject to subject. If the amplitude of a diastolic murmur peak is large enough, it can be picked up as a possible candidate for an S₁ or S₂ peak. The two previously described time domain and frequency domain kurtosis calculations distinguish the S₁ and S₂ peaks from the diastolic murmur (900) peaks. FIG. 9 shows that despite the fact that the diagnostic rumble (900) peaks are comparable to S₁ and S₂ peaks in amplitude, the methods described herein are able to correctly estimate the locations of the S₁ and S₂ peaks.

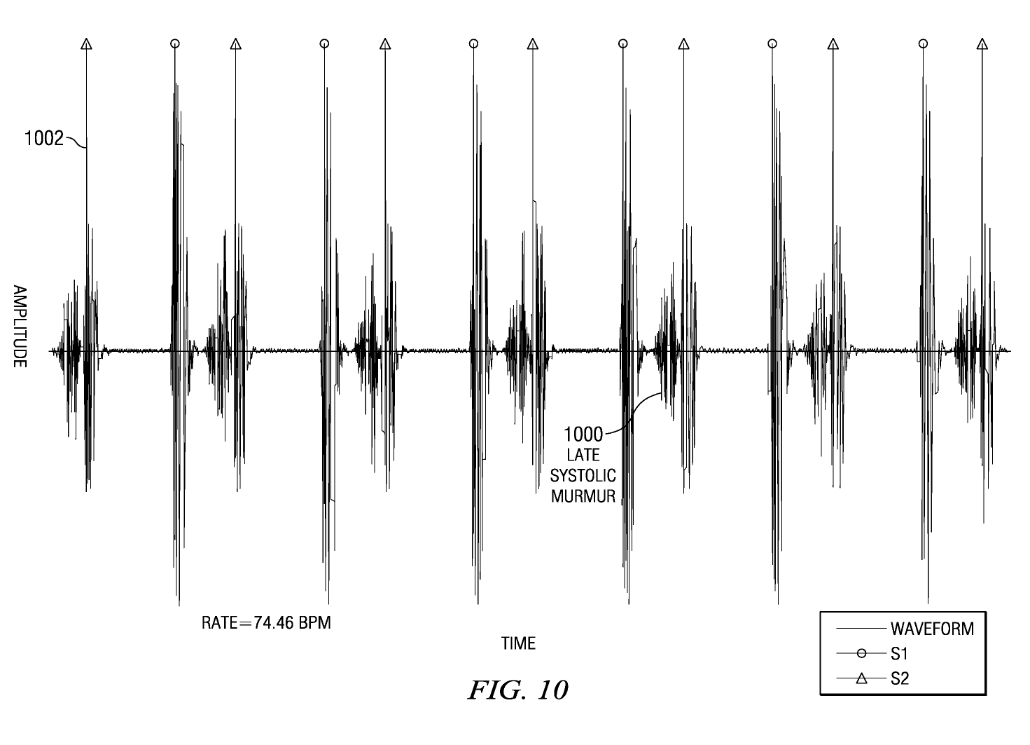

FIG. 10 shows the result of analyzing an audio signal of heart sounds that include a late systolic murmur (1000). The systolic murmur sound occurs between S₁ and S₂. Further, if a late systolic murmur is present, the sound may occur quit close to the S₂ sound and can be confused for the S₂ sound. The previously described frequency domain kurtosis calculations distinguish the S₂ peaks from the late systolic murmur peaks (1000). Note that in this particular audio signal, the primary heart sound encountered first ((1002) was an S₂ sound, but this sound was not misinterpreted as an S₁ sound. This is due to the distance check between a previous S1 peak and an S₂ peak and between the S₂ peak and the subsequent S₁ peak as described above.

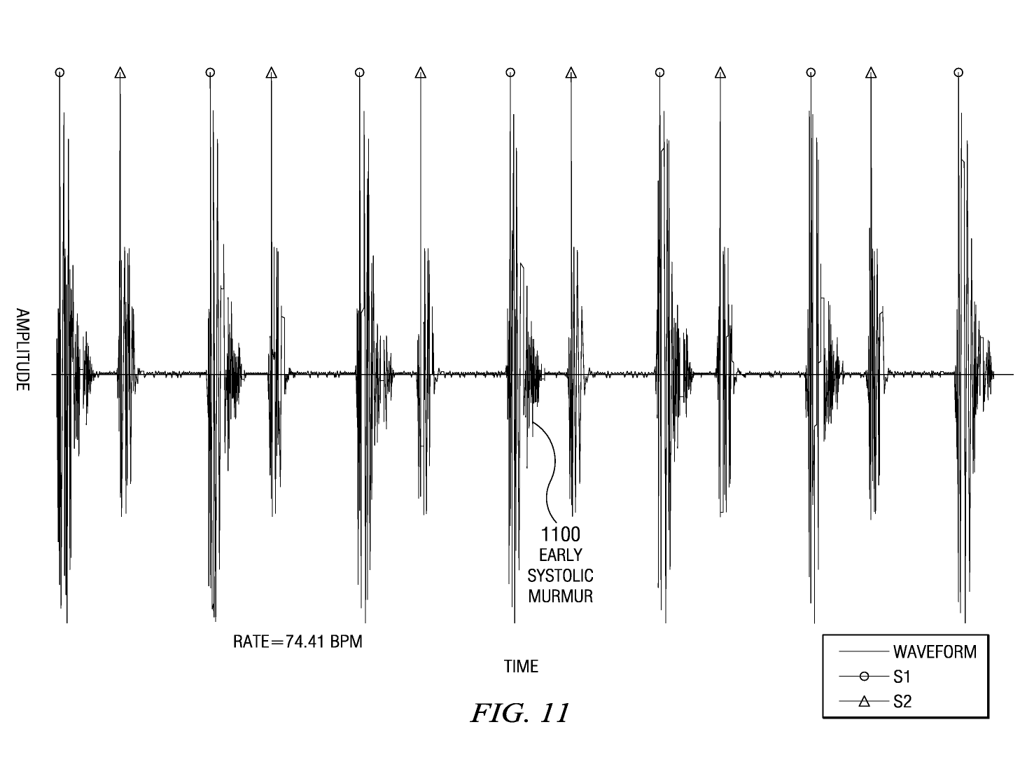

FIG. 11 shows the result of analyzing an audio signal of heart sounds that include an early systolic murmur (1100). Early systolic murmur sounds generally have amplitude lower than that of S₁ sounds and do not interfere with locating the S₁ peak. In cases where the amplitude of early systolic murmur peaks is comparable to that of S₁ peaks, the previously described time domain kurtosis calculations distinguish the S₁ peaks from the early systolic murmur peaks (1100).

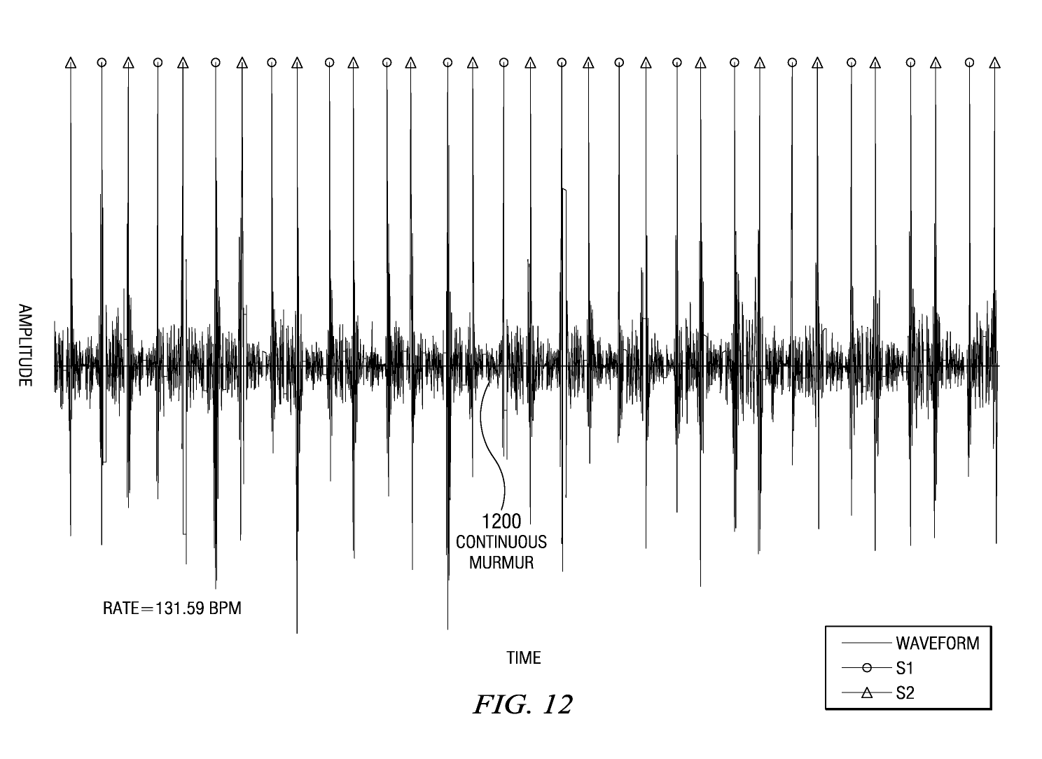

FIG. 12 shows the result of analyzing an audio signal of heart sounds that include a continuous murmur (1200). A continuous murmur (1200) increased the difficulty of locating S₁ and S₂ peaks as it corrupts the S₁ and S₂ sounds. The previously discussed timing based error correction distinguishes S₁ and S₂ peaks from continuous murmur (1200) peaks.

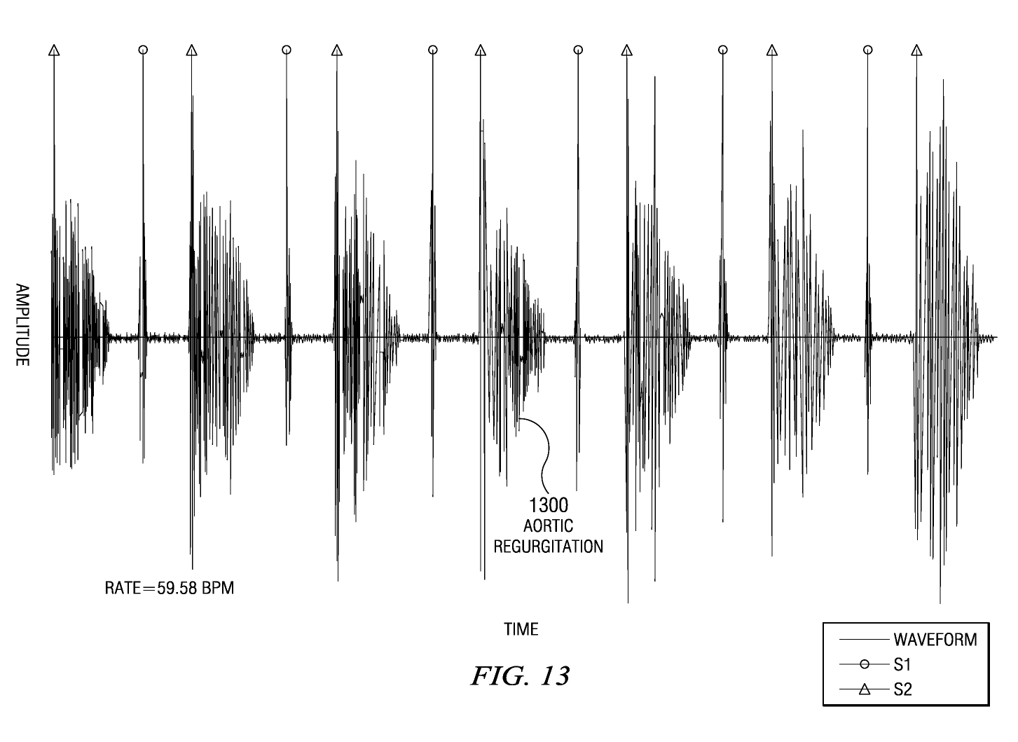

FIG. 13 shows the result of analyzing an audio signal of heart sounds that include aortic regurgitation (AR) (1300). Mild AR usually does not interfere with locating S₂ peaks. However, as can be seen in FIG. 13, it is possible for AR (1300) peaks to mask S₂ peaks. When sufficient AR (1300) is present, the analysis initially will not find a legitimate S₂ peak between two S₁ peaks, and instead estimates the location of the S₂ peak to be the highest peak between the two S₁ peaks. The previously discussed timing based error correction ensures that either this estimate is the location of the S₂ peak or that a nearby peak is the S₂ peak.

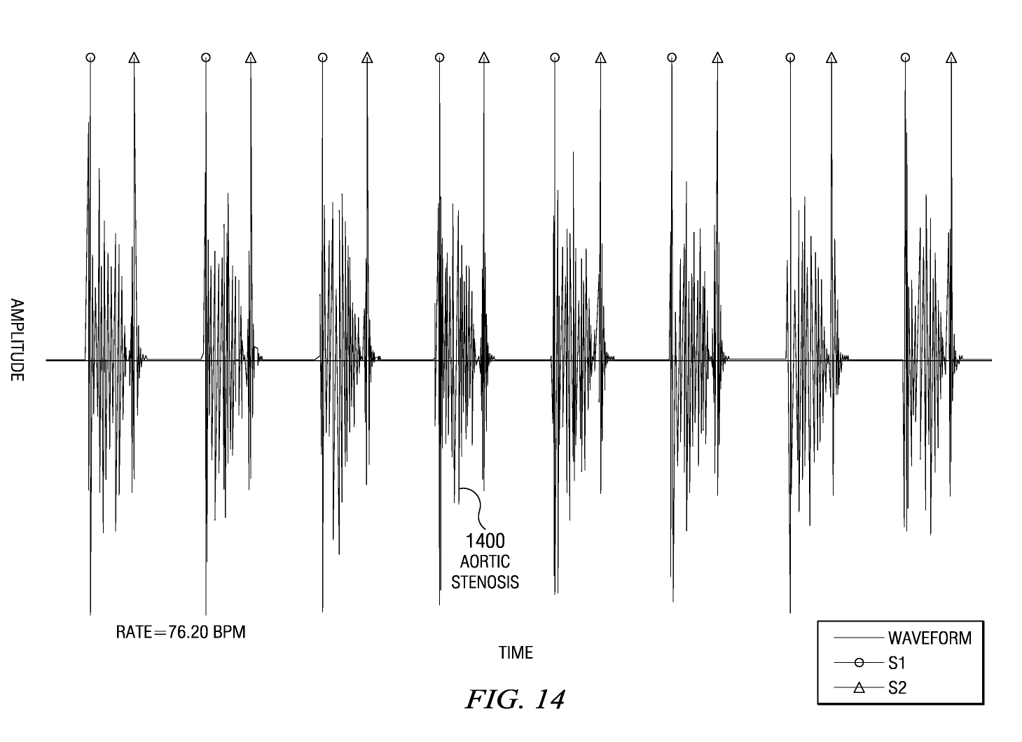

FIG. 14 shows the result of analyzing an audio signal of heart sounds that include aortic stenosis (AS) (1400). Mild AS usually does not interfere with locating S₁ peaks. However, as can be seen in FIG. 14, it is possible for AS (1400) peaks to mask S₁ peaks. The analysis still correctly locates S₁ peaks due to the fact that S₁ peaks will usually occur before the AS (1400) peaks. More specifically, the analysis initially identifies the highest peak in a search window as the S₁ peak. Then, the previously discussed timing based error correction ensures that either this peak or a nearby peak is the S₁ peak.

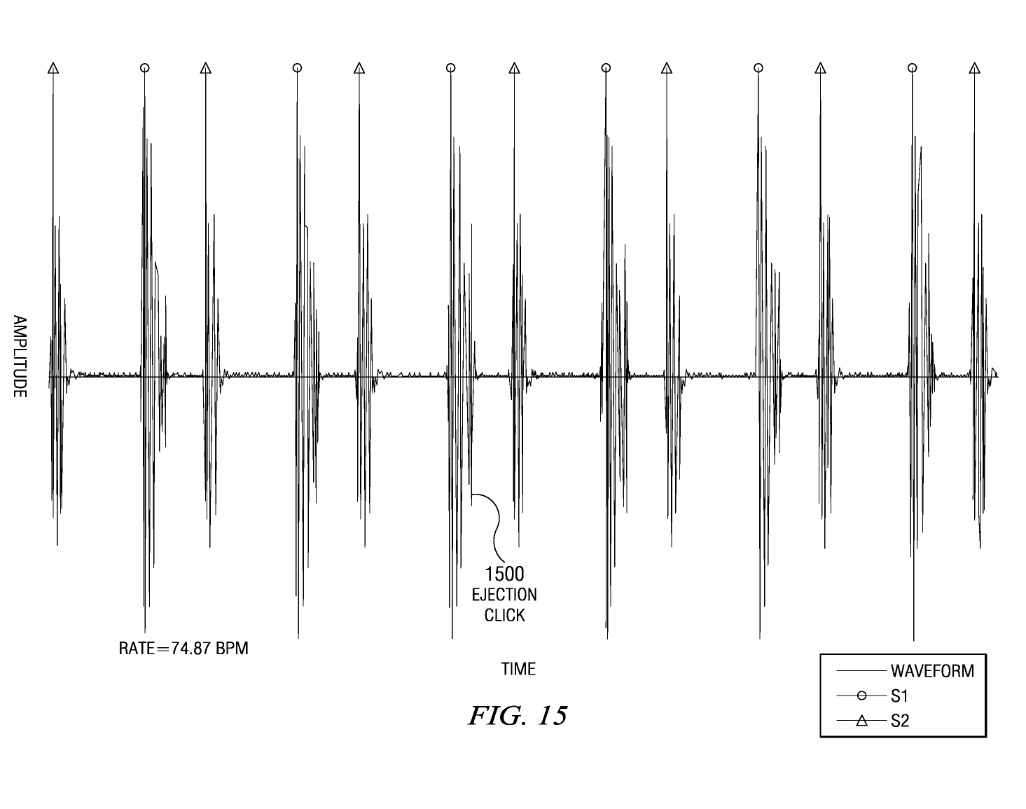

FIGS. 15-18 show PCGs of the results of analyzing audio signals with heart sounds that include other abnormal cardiac conditions. These figures illustrate the ability to distinguish the primary heart sounds, S₁ and S₂, from heart sounds introduced by these abnormalities. FIG. 15 shows the result of analyzing an audio signal of heart sounds that include ejection clicks (1500). Ejection clicks occur very close to S₁ sounds and are smaller in amplitude and hence are usually easily eliminated during the analysis. However, in some cases, ejection clicks (1500) can cause the kurtosis measures of S₁ peaks to resemble those of a murmur. In such cases, the previously discussed timing based error correction ensures that the S₁ peak is located.

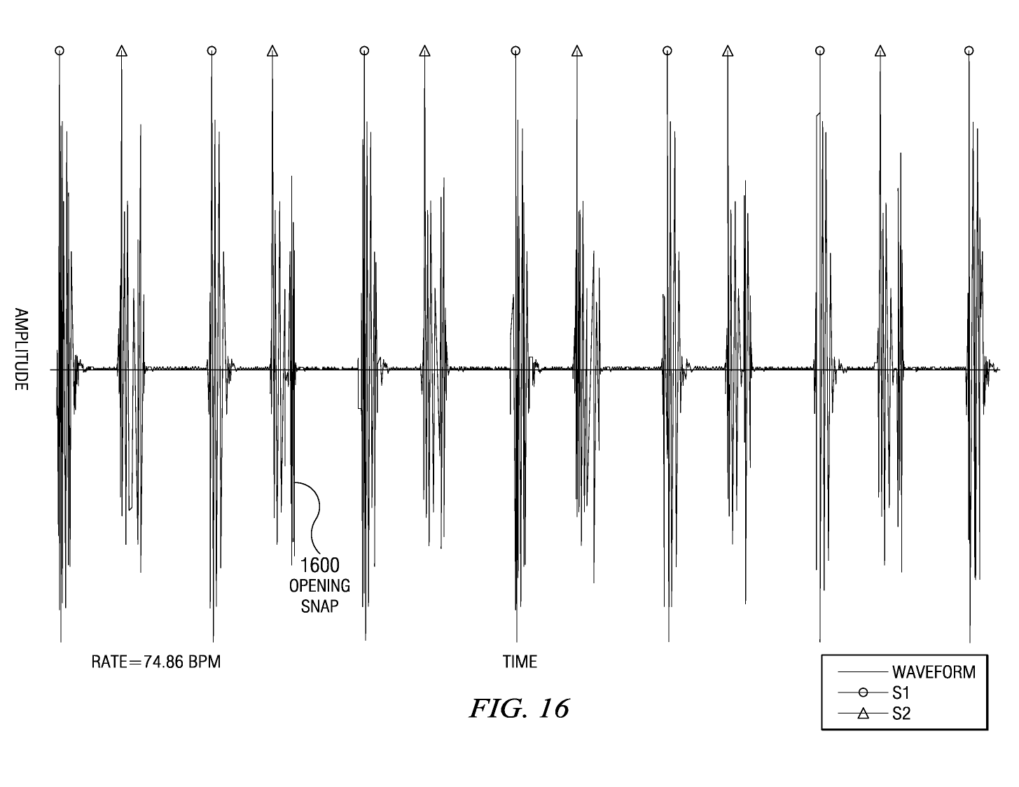

FIG. 16 shows the result of analyzing an audio signal of heart sounds that include opening snaps (1600). Opening snaps occur very close to S₂ sounds and sometimes have amplitude greater than that of an S₂ peak. In the analysis, once a location is identified as a possible S₂ peak, errors due to opening snaps are eliminated by testing for “real” S₂ peak locations before the currently identified location. In addition, opening snap (1600) peaks are distinguished from S₃ peaks by exploiting the fact that an opening snap peak (1600) occurs temporally much closer to an S₂ peak than does an S₃ peak.

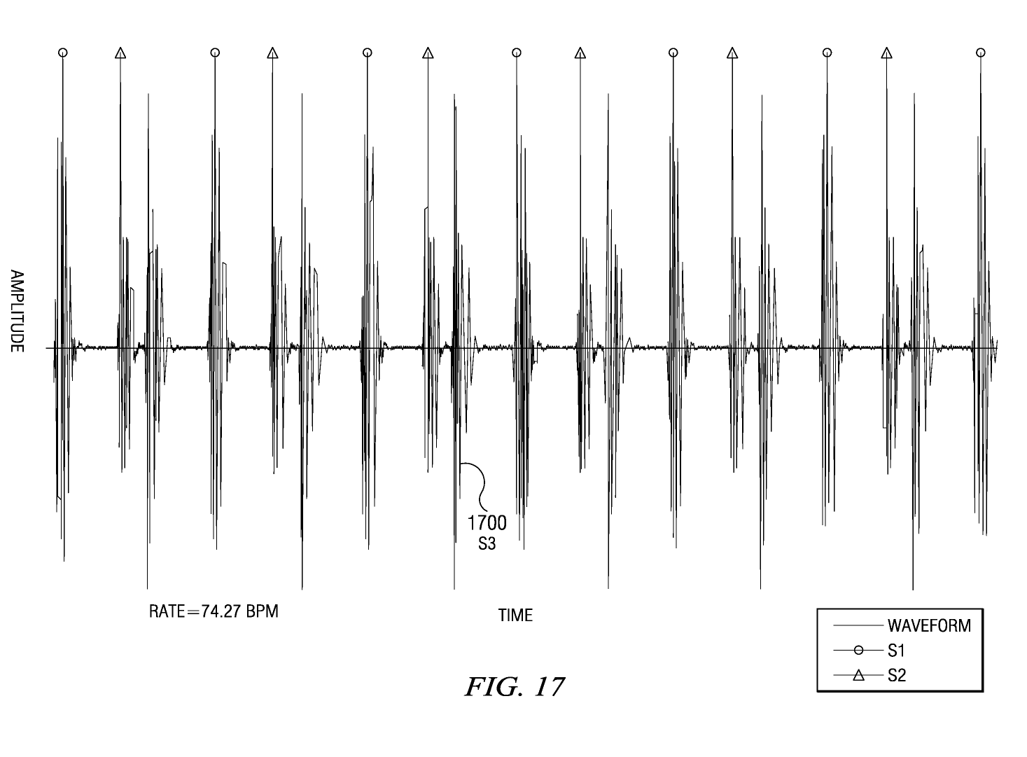

FIG. 17 shows the result of analyzing an audio signal of heart sounds that include S₃ (1700). As can be seen in FIG. 17, S₃ (1700) can have amplitude much larger than S₂. Spectrally, S₂ and S₃ sounds are similar; hence it is easy to confuse S₃ peaks for S₂ peaks. In the analysis, once a location is identified as a possible S₂ peak, errors due to S₃ are eliminated by testing for “real” S₂ peak locations before the currently identified location. As previously described, this testing locates a peak within a predetermined distance of the possible S₂ peak with sufficient amplitude to be an S₂ peak, if one is present. If such a peak is not present, the possible S₂ peak is the real S₂ peak. If such a peak is present, this new peak is checked with frequency based kurtosis to eliminate the possibility that the new peak is a late systolic murmur. If the new peak is a late systolic murmur, then the possible S₂ peak is the real S₂ peak; otherwise the new peak is the real S₂ peak and the possible S₂ peak is an S₃ peak.

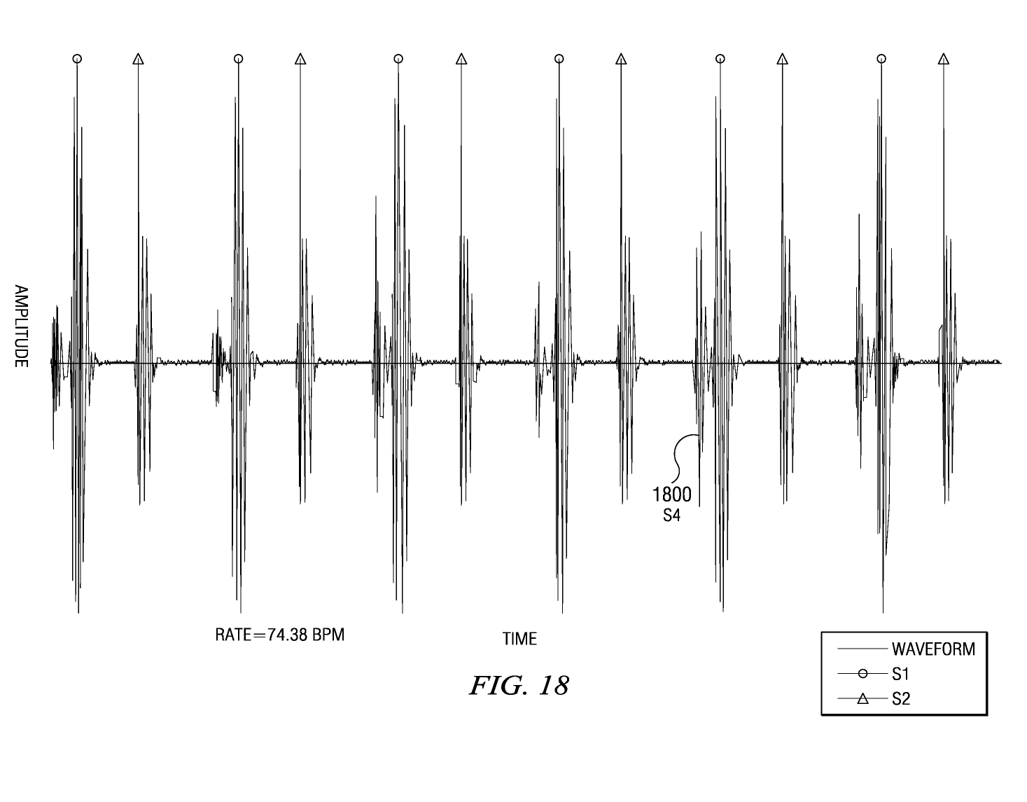

FIG. 18 shows the result of analyzing an audio signal of heart sounds that include S₄ (1800). S₄ peaks occur just before S₁ peaks and are generally much smaller in amplitude than S₁. S₄ peaks do not usually interfere with detection of S₁ peaks.

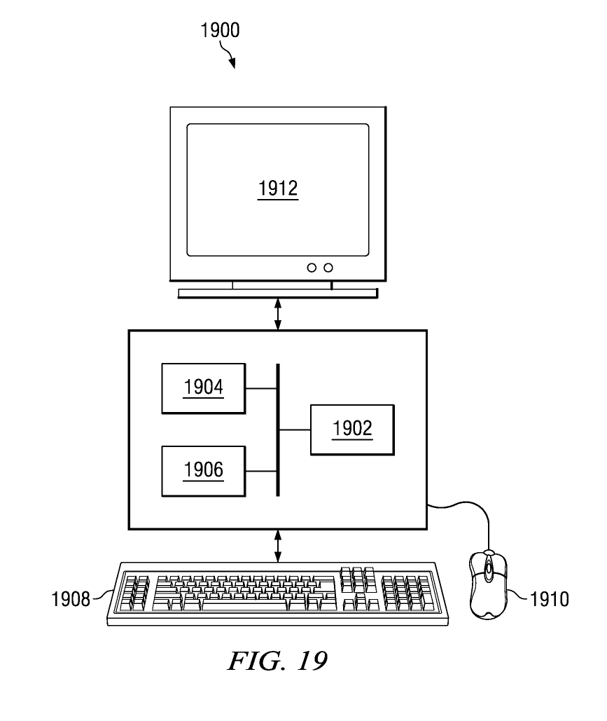

Embodiments of the methods described herein may be implemented on virtually any type of computing system. For example, as shown in FIG. 19, a computer system (1900) includes a processor (1902), associated memory (1904), a storage device (1906), and numerous other elements and functionalities typical of today's computing systems (not shown). The computer system (1900) may also include input means, such as a keyboard (1908) and a mouse (1910) (or other cursor control device), and output means, such as a monitor (1912) (or other display device). The computer system (1900) may be connected to a network (1914) (e.g., a local area network (LAN), a wide area network (WAN) such as the Internet, or any other similar type of network) via a network interface connection (not shown). Those skilled in the art will appreciate that these input and output means may take other forms.

Further, those skilled in the art will appreciate that one or more elements of the aforementioned computer system (1900) may be located at a remote location and connected to the other elements over a network. Further, embodiments of the invention may be implemented on a distributed system having a plurality of nodes, where each portion of the system and software instructions may be located on a different node within the distributed system. In one embodiment of the invention, the node may be a computer system. Alternatively, the node may be a processor with associated physical memory. The node may alternatively be a processor with shared memory and/or resources. Further, software instructions to perform embodiments of the invention may be stored on a computer readable medium such as a compact disc (CD), a diskette, a tape, a file, or any other computer readable storage device.

While the invention has been described with respect to a limited number of embodiments, those skilled in the art, having benefit of this disclosure, will appreciate that other embodiments can be devised which do not depart from the scope of the invention as disclosed herein. Accordingly, the scope of the invention should be limited only by the attached claims.

It is therefore contemplated that the appended claims will cover any such modifications of the embodiments as fall within the true scope and spirit of the invention.

What is claimed is:

1. A method for identification of heart sound components comprising:
receiving an audio signal comprising heart sounds;
identifying a first peak corresponding to S₁ within a first search window of the audio signal, wherein identifying the first peak comprises distinguishing the first peak from a murmur peak using time domain kurtosis;
identifying a second peak corresponding to S₁ within a second search window of the audio signal, wherein identifying the second peak comprises distinguishing the second peak from a murmur peak using time domain kurtosis;
identifying a third peak corresponding to S₂ between the first peak and the second peak, wherein identifying the third peak comprises using frequency domain kurtosis to determine whether another peak that may be the third peak is a murmur peak; and
storing a location of the first peak as a first S₁ location, storing a location of the second peak as a second S₁ location, and storing a location of the third peak as an S₂ location.

2. The method of claim 1, further comprising:
verifying that a first distance between the first peak and the third peak is smaller than a second distance between the third peak and the second peak.

3. The method of claim 1, further comprising:
locating a fourth peak and a fifth peak that may correspond to S₁ and S₂ between the first peak and the third peak; and
distinguishing the fourth peak and the fifth peak from murmurs using time domain kurtosis.

4. The method of claim 3, further comprising:
when the fourth peak and the fifth peak correspond to S₁ and S₂,
reducing a size of the first search window;
reducing a length between the first peak identified in the first search window and a beginning of the second search window; and
repeating the identifying a first peak, the identifying a second peak, and the identifying a third peak.

5. The method of claim 1, further comprising:
performing timing based error correction to verify that the second peak corresponds to S₁ and the third peak corresponds to S₂.

6. The method of claim 5, wherein performing timing based error correction comprises:
when a distance between the first peak and the second peak is not within a predetermined percentage of an average distance between two consecutive S₁ peaks, locating another peak corresponding to S₁, wherein a distance between the first peak and the another peak is within the predetermined percentage of the average distance, and identifying the another peak as the second peak.

7. The method of claim 5, wherein performing timing based error correction comprises:
when a distance between the third peak and a fourth peak corresponding to S₂ is not within a predetermined percentage of an average distance between two consecutive S₂ peaks, locating another peak corresponding to S₂, wherein a distance between the third peak and the another peak is within the predetermined percentage of the average distance, and identifying the another peak as the third peak.

8. The method of claim 5, wherein performing timing based error correction comprises:
when a distance between the first peak and the third peak is not within a predetermined percentage of an average distance between an S₁ peak and a subsequent S2 peak, locating another peak corresponding to S₂, wherein a distance between the first peak and the another peak is within the predetermined percentage of the average distance, and identifying the another peak as the third peak.

9. The method of claim 5, wherein performing timing based error correction comprises:
when a distance between the third peak and the second peak is not within a predetermined percentage of an average distance between an S₂ peak and a subsequent S₁ peak, locating another peak corresponding to S₁, wherein a distance between the third peak and the another peak is within the predetermined percentage of the average distance, and identifying the another peak as the second peak.

10. The method of claim 1, further comprising:
determining a heart rate using the stored S₁ locations.

11. The method of claim 1, wherein distinguishing the first peak from a murmur further comprises:
computing a first kurtosis of a segment of the audio signal that is a predetermined number of milliseconds on either side of the first peak;
computing a second kurtosis of a segment of the audio signal that is the predetermined number of milliseconds before the first peak; and
computing a third kurtosis of a segment of the audio signal that is the predetermined number of milliseconds after the second peak,
wherein when the first kurtosis is greater than a first predetermined value or an absolute difference between the second kurtosis and the third kurtosis is greater than a second predetermined value, the first peak is not a murmur.

12. The method of claim 1, wherein using frequency domain kurtosis further comprises:
computing a first kurtosis of a magnitude of a Fourier transform of a segment of the audio signal beginning at a location of the third peak; and
computing a second kurtosis of a magnitude of a Fourier transform of a segment of the audio signal beginning at a location of the another peak,
wherein a length of the segments is the nearest power of two to the length in samples that equals 50 ms, and
wherein when an absolute difference between a geometric mean of the first kurtosis and the second kurtosis and an arithmetic mean of the first kurtosis and the second kurtosis is greater than a predetermined value and the first kurtosis is greater than the second kurtosis, the another peak is determined to be a murmur peak.

13. The method of claim 1, wherein
distinguishing the first peak from a murmur further comprises:
identifying a murmur; and
storing a location of the murmur, and
wherein the location of the murmur and the stored S₁ locations and stored S₂ location are used to determine a type of the murmur.

14. The method of claim 5, wherein performing timing based error correction further comprises identifying a new peak as the second peak, wherein identifying the new peak as the second peak indicates a possible murmur interfering with the second peak.

15. The method of claim 5, wherein performing timing based error correction further comprises identifying a new peak as the third peak, wherein identifying the new peak as the third peak indicates a possible murmur interfering with the third peak.

16. A system comprising:
a processor;
a display operatively connected to the processor;
a memory operatively connected to the processor; and
instructions stored in the memory that are executable by the processor to identify heart sound components by:
receiving an audio signal comprising heart sounds;
identifying a first peak corresponding to S₁ within a first search window of the audio signal, wherein identifying the first peak comprises distinguishing the first peak from a murmur peak using time domain kurtosis;
identifying a second peak corresponding to S₁ within a second search window of the audio signal, wherein identifying the second peak comprises distinguishing the second peak from a murmur peak using time domain kurtosis;
identifying a third peak corresponding to S₂ between the first peak and the second peak, wherein identifying the third peak comprises using frequency domain kurtosis to determine whether another peak that may be the third peak is a murmur peak; and
storing a location of the first peak as a first S₁ location, storing a location of the second peak as a second S₁ location, and storing a location of the third peak as an S₂ location,
wherein the first S₁ location, the second S₁ location, and the S₂ location are shown in a phonocardiogram on the display.

17. The system of claim 16, wherein the instructions further identify heart sound components by:
performing timing based error correction to verify that the second peak corresponds to S₁ and the third peak corresponds to S₂.

18. The system of claim 16, wherein the system is one selected from a group consisting of a digital stethoscope, a personal computer, a laptop computer, a server, a mainframe, a personal digital assistant, a mobile phone, an iPod, and an MP3 player.

19. A computer readable medium storing instructions for identifying heart sound components, the instructions comprising functionality for:
receiving an audio signal comprising heart sounds;
identifying a first peak corresponding to S₁ within a first search window of the audio signal, wherein identifying the first peak comprises distinguishing the first peak from a murmur peak using time domain kurtosis;
identifying a second peak corresponding to S₁ within a second search window of the audio signal, wherein identifying the second peak comprises distinguishing the second peak from a murmur peak using time domain kurtosis;
identifying a third peak corresponding to S₂ between the first peak and the second peak, wherein identifying the third peak comprises using frequency domain kurtosis to determine whether another peak that may be the third peak is a murmur peak; and
storing a location of the first peak as a first S₁ location, storing a location of the second peak as a second S₁ location, and storing a location of the third peak as an S₂ location.

20. The computer readable medium of claim 19, wherein the instructions further comprise functionality for:
performing timing based error correction to verify that the second peak corresponds to S₁ and the third peak corresponds to S₂.
